(19) United States
(12) Patent Application Publication
(10) Pub. No.: US 2016/0319232 A1
(43) Pub. Date: Nov. 3, 2016

(54) MEASURING CELL, DETECTOR, AND ANALYSIS DEVICE

(71) Applicant: Kabushiki Kaisha Toshiba, Minato-ku (JP)

(72) Inventors: Yasuko NORITOMI, Kawasaki (JP); Takashi KUBOKI, Tokyo (JP); Ko YAMADA, Yokohama (JP); Hiroyuki FUKE, Yokohama (JP)

(73) Assignee: Kabushiki Kaisha Toshiba, Minato-ku (JP)

(21) Appl. No.: 15/139,832

(22) Filed: Apr. 27, 2016

(30) Foreign Application Priority Data
Apr. 30, 2015 (JP) 2015-093553

(51) Int. Cl. C12M 1/40; G01N 27/403

(52) U.S. Cl. CPC C12M 21/18; G01N 27/403

(57) ABSTRACT

According to one embodiment, a measuring cell includes a main cell member, and a mixture supported by or held in the main cell member. The mixture includes a nonaqueous solvent-including medium and one or more enzyme bodies. The one or more enzyme bodies are selected from the group including an enzyme, a first composite including an enzyme and a molecular aggregate that includes a dispersant, a microcapsule including an enzyme-including core and a shell covering the core, a cell including an enzyme, a microorganism including an enzyme, and a second composite including an enzyme and a support immobilizing the enzyme.

### CROSS-REFERENCE TO RELATED APPLICATIONS

This application is based upon and claims the benefit of priority from Japanese Patent Application No. 2015-093553 filed Apr. 30, 2015; the entire contents of which are incorporated herein by reference.

### FIELD

Embodiments described herein generally relate to a measuring cell, detector, and analysis device.

### BACKGROUND

A detector equipped with an electrochemical sensor may be used to detect a gas sample or liquid sample. Such a detector is equipped with, e.g., an electrolyte solution, and detects a measurement target substance included in a sample to be measured by measuring the electrochemical property of the electrolyte solution using an electrode before and after the sample to be measured is introduced.

Of detectors equipped with electromechanical sensors, an enzyme sensor type detector makes use of a chemical reaction catalyzed by an enzyme. In this enzyme sensor type detector, a reaction product formed by a reaction catalyzed by an enzyme, i.e., an enzyme reaction affects the electrochemical property of an electrolyte solution.

Many enzymes show specific enzyme activity with respect to a specific measurement target substance. The use of such an enzyme makes it possible to obtain an enzyme sensor type detector capable of detecting a specific measurement target substance.

### BRIEF DESCRIPTION OF THE DRAWINGS

FIG. 1 is a schematic view of an example of a detector according to the first embodiment;

FIGS. 2A and 2B are graphs each showing an example of a measurement mode obtained by electrical measurement or electrochemical measurement according to the embodiment;

FIG. 3 is a schematic view of another example of the detector according to the first embodiment;

FIG. 4 is a schematic view of an example of a detector according to the second embodiment;

FIG. 5 is a flowchart showing procedures of measurement and alarm transmission by the detector according to the embodiment;

FIG. 6 is a schematic view of a specific example of the detector according to the first embodiment;

FIGS. 7A, 7B, 7C, and 7D are schematic views of a specific example of a measuring cell according to the first embodiment;

FIGS. 8A and 8B are schematic views of other specific examples of the detector according to the first embodiment;

FIGS. 9A and 9B are schematic views of still other examples of the detector and measuring cell according to the first embodiment;

FIGS. 10A, 10B, 10C, and 10D are schematic views of still other examples of the detector according to the first embodiment; and

FIGS. 11A and 11B are schematic views of still other examples of the detector according to the first embodiment.

### DETAILED DESCRIPTION

According to one embodiment, a measuring cell includes a main cell member, and a mixture supported by or held in the main cell member. The mixture includes a nonaqueous solvent-including medium and one or more enzyme bodies. The one or more enzyme bodies are selected from the group including an enzyme, a first composite including an enzyme and a molecular aggregate that includes a dispersant, a microcapsule including an enzyme-including core and a shell covering the core, a cell including an enzyme, a microorganism including an enzyme, and a second composite including an enzyme and a support immobilizing the enzyme.

According to another embodiment, a detector includes the abovementioned measuring cell, and a measuring unit configured to measure an electrical property or an electrochemical property of the mixture. The measuring cell further includes one or more electrodes disposed in contact with the mixture.

According to yet another embodiment, a detector includes the abovementioned measuring cell, and a measuring unit configured to measure an optical characteristic of the mixture.

According to still another embodiment, an analysis device includes the abovementioned detector and a sampling unit. The sampling unit includes at least one of a vaporizer and an ionization source. The vaporizer is configured to vaporize a measurement target substance included in a sample to be measured by laser irradiation, UV irradiation, gas spraying, ultrasonic irradiation, heating, or voltage application. The ionization source is configured to ionize the measurement target substance.

The embodiments will be explained in detail below with reference to the accompanying drawings. In the description of the following drawings, the same or similar reference numerals denote the same or similar parts. However, it should be noted that these drawings are schematic views, and the ratios of dimensions and the like are different from those in reality. Accordingly, practical dimensions and the like should be judged by referring to the following explanation. Also, the drawings include portions where the relationships and ratios of dimensions are different between drawings.

### First Embodiment

A measuring cell according to an embodiment includes a main cell member, and a mixture supported by or held in the main cell member. This mixture includes a nonaqueous solvent-including medium and one or more enzyme bodies. The enzyme body includes an enzyme, and details will be described later.

When a substrate is introduced to the mixture of the measuring cell according to the embodiment, a reaction catalyzed by the enzyme included in the enzyme body, i.e., an enzyme reaction proceeds. As a result, the electrical property, electrochemical property, or optical property of the mixture changes. In this embodiment, a measurement target substance itself is a substrate which reacts by the enzyme reaction. That is, a measurement target substance included in a sample to be measured can be detected by measuring the change in electrical, electrochemical, or optical property of the mixture when the measurement target substance is introduced.

A detector according to the embodiment includes the above-described measuring cell, and a measuring unit for measuring the electrical, electrochemical, or optical property of the mixture included in the measuring cell. When measuring the electrical or electrochemical property of the mixture included in the measuring cell, the detector further includes one or more electrodes disposed in contact with the mixture. When measuring the optical characteristic of the mixture, the detector may further include a dye.

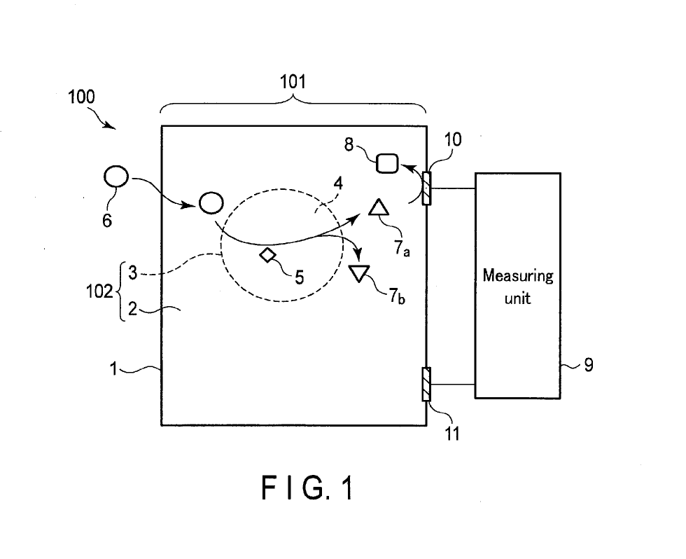

FIG. 1 is a schematic view of an example of the detector according to the embodiment.

A detector 100 shown in FIG. 1 includes a measuring cell 101 that includes a mixture 102 including a nonaqueous solvent-including medium 2 and an enzyme body 3, and a measuring unit 9. The detector 100 shown in FIG. 1 further includes a pair of electrodes including a detection electrode 10 and comparison electrode 11 as working electrodes.

FIG. 1 shows one pair of electrodes, but the number of electrodes may be one or two or more as will be described later.

The measuring cell 101 may be detachable from the detector 100. In this case, when attaching the measuring cell 101 to the detector 100, the one or more electrodes of the measuring cell 101 may be electrically connected to the measuring unit 9, or the measuring cell 101 and measuring unit 9 may be connected wirelessly.

In the detector 100 shown in FIG. 1, the enzyme bodies 3 are dispersed in the medium 2 in vicinity of the detection electrode 10. By contrast, no enzyme bodies 3 are dispersed in the medium 2 in vicinity of the comparison electrode 11.

In the detector 100 shown in FIG. 1, the mixture 102 includes the enzyme bodies 3 and medium 2. Here, the enzyme bodies 3 are only of one type, and each enzyme body 3 includes one kind of enzyme 5. The mixture 102 is supported by or held in the main cell member 1 of the measuring cell 101. The enzyme body 3 includes water, and this water forms a water pool 4.

In FIG. 1, when the enzyme reaction in the enzyme body 3 catalyzed by the enzyme 5 is an enzyme reaction requiring water, such as hydrolysis, the water of the water pool 4 included in the enzyme body 3 can be used for the enzyme reaction. Also, the enzyme 5 shows high activity because the water pool 4 serves as the reaction field of the enzyme reaction.

The nonaqueous solvent included in the medium 2 of the mixture 102 may be a nonaqueous solvent which in itself functions as an electrolyte, e.g., an ionic liquid. When such a nonaqueous solvent is used, it becomes unnecessary to dissolve another electrolyte in the medium 2. In addition, the concentration of the electrolyte solution remains unchanged, and precipitation of the electrolyte does not occur. Furthermore, the measuring cell 101 including the mixture 102 can be used over a long period of time because the nonaqueous solvent hardly evaporates.

Details of the enzyme body 3 and medium 2 which compose the mixture 102 will be described later.

In the detector 100, a measurement target substance 6 itself included in a sample to be measured is a substrate. When the measurement target substance 6 is introduced to the mixture 102 of the measuring cell 101, the enzyme reaction of the measurement target substance 6 as a substrate proceeds due to the catalytic action of the enzyme 5 of the enzyme body 3, thereby forming one or more products. For example, suppose that products 7a and 7b are formed. The measuring unit 9 detects a change in electrical or electrochemical property of the mixture 102 caused by this, as an electrical signal via the detection electrode 10, thereby detecting the measurement target substance 6.

When at least one of the products 7a and 7b is a substance such as a redox species which participates a redox reaction on the surface of electrode, i.e., an electrode active material, the change in electrochemical property of the mixture 102 can be measured. In this example, it is supposed that the product 7a is an electrode active material.

For the measurement of change in electrochemical property, voltammetry may be used, for example. When measuring the change in electrochemical property by voltammetry, for example electrochemical measurement methods such as cyclic voltammetry (CV), amperometry, chronoamperometry (CA), alternate current voltammetry (AC voltammetry), potential-step voltammetry, stepwise-wave voltammetry, pulse voltammetry, and chronopotentiometry may be used.

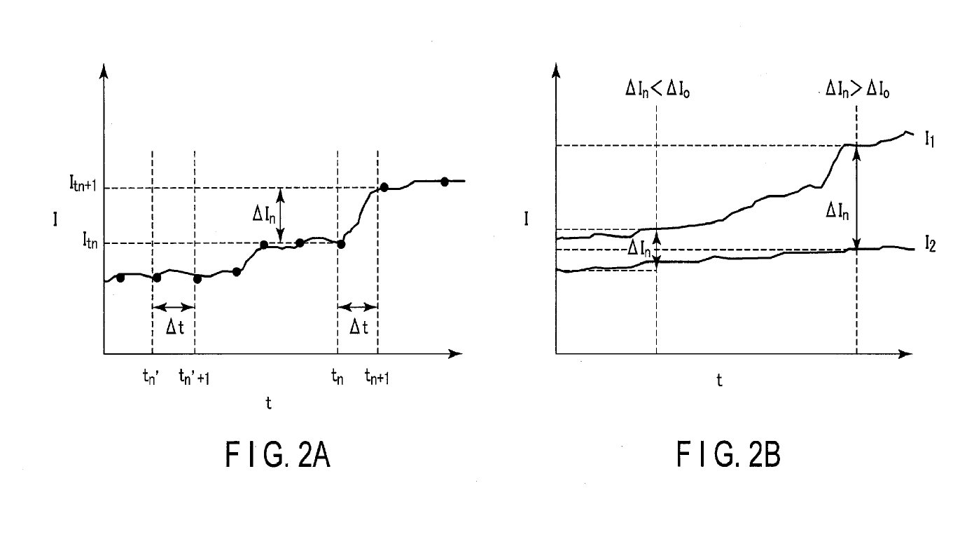

When the detector 100 includes only the detection electrode 10 as a working electrode, for example a change in oxidation current or reduction current with time may be measured using chronoamperometry (CA) by a measurement mode (S1 measurement mode) as such as shown in FIG. 2A.

More specifically, in the S1 measurement mode, the measurement target substance 6 can be detected by measuring a change in value of an electric current flowing through the electrode 10 in a set time interval (Δt=tₙ₊₁−tₙ) from first time (tₙ) to second time (tₙ₊₁) i.e., a difference (ΔIₙ) between a current value (Iₜₙ) at first time (tₙ) and a current value (Iₜₙ₊₁) at second time (tₙ₊₁) (Equation 1):

ΔIₙ=|Iₜₙ₊₁−Iₜₙ| (Equation 1)

In a state in which no sample to be measured is introduced to the measuring cell 101, i.e., in a steady state, it is possible to obtain current values (I_{tn′} and I_{tn′+1}) at first time (t_{n′}) and second time (t_{n′+1}), and define the difference (ΔIₒ=|I_{tn′+1}−I_{tn′}|) between these current values as a noise-level current change value in advance.

When the detector 100 includes not only the detection electrode 10 but also the comparison electrode 11 as working electrodes as shown in FIG. 1, a measurement mode as shown in FIG. 2B may be used. This measurement mode is referred to as an S2 measurement mode hereinafter. In this S2 measurement mode, the measurements by the CA method are performed using both the detection electrode 10 and comparison electrode 11 at the same time. Then, the sample to be measured is detected based on the difference (ΔIₙ=|I₁−I₂|) between the current value (I₁) of the detection electrode 10 and the current value (I₂) of the comparison electrode 11 obtained at the same time. In the S2 measurement mode, in a steady state in which no sample to be measured is introduced to the measuring cell 101, it is possible to obtain the difference (ΔIₙ=|I₁−I₂|) between the current value (I₁) of the detection electrode 10 and the current value (I₂) of the comparison electrode 11, and define this difference as a noise-level current change value (ΔIₒ).

When performing measurement, for example by the S2 measurement mode in the detector shown in FIG. 1, the concentration of the measurement target substance 6 in the mixture 102 increases, and the concentration of the product 7a increases accordingly. Since the detection electrode 10 detects an oxidation current or reduction current of the product 7a, the current value (I₁) of the detection electrode 10 increases. On the other hand, since no enzyme bodies 3 are dispersed in vicinity of the comparison electrode 11, neither an oxidation current nor a reduction current of the product 7a is detected at the comparison electrode 11. That is, the current value (I₂) of the comparison electrode 11 is held constant. Consequently, the current value difference (ΔIₙ) associated with oxidation or reduction of the product 7a is larger than the noise-level current change value (ΔIₒ).

When performing quantitative measurement of a sample to be measured by the above-described method, the relationship between a current change amount and the concentration of the sample to be measured may be confirmed beforehand. For example, a database constructed by forming a calibration curve may be stored in a data processor of the measuring unit 9. Note that the measuring unit 9 can have not only functions of calculating and outputting data, but also functions of controlling measurement conditions, exchanging data, and sending an alarm. Note also that the connection between the measuring cell 101 and measuring unit 9 may be either wired or wireless.

When the measuring cell 101 and measuring unit 9 are wirelessly connected, each of the measuring cell 101 and measuring unit 9 has a wireless transmitting/receiving function. When performing wireless communication, for example by an electromagnetic field or radio wave as with an RFID (Radio Frequency IDentification), a passive tag may be attached to the measuring cell 101 as a member having a receiving function. Also, a reader may be attached to the measuring unit 9 as a member having a transmitting function. The passive tag for use in the RFID can operate by using, as an energy source, the radio wave transmitted by the reader. When the RFID using the passive tag is adopted, therefore, the measuring cell 101 need not have a battery built-in. The radio wave received from the reader by the passive tag can be used as electric energy for measurement in the measuring cell 101 and for transmitting and receiving data.

Detection by CA measurement has been explained as an example of the method of detecting the measurement target substance 6 by electrochemical measurement using the detector 100; however, the electrochemical measurement method is not limited to this. Also, the design of the detector 100 may be changed in accordance with an electrochemical measurement method to be adopted. Various electrochemical measurement methods and the design of the detector 100 corresponding to the adopted method will be described in detail later.

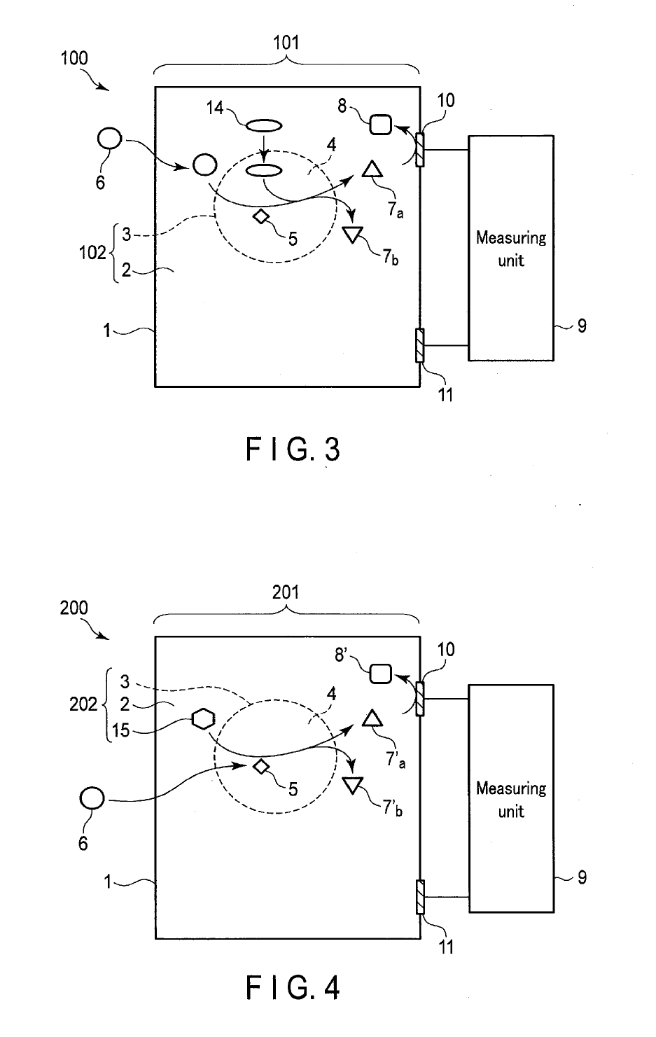

The detector 100 shown in FIG. 3 has the same arrangement as that of the detector 100 shown in FIG. 1 except that a mediator 14 is included.

In the detector 100 shown in FIG. 3, both the measurement target substance 6 as a substrate and the mediator 14 participate in the enzyme reaction in the enzyme body 3. For example, when the measurement target substance 6 is oxidized or reduced by the enzyme reaction, the mediator 14 is reduced or oxidized by the enzyme reaction accordingly, and the products 7a and 7b are formed.

The measuring unit 9 detects a change in electrical or electrochemical property of the mixture 102 caused by the formation of the products 7a and 7b, as an electrical signal via the working electrode 10, thereby detecting the measurement target substance 6. For example, the product 7a forms a redox product 8 by an oxidation or reduction reaction at the detection electrode 10. The measuring unit 9 detects an electric current generated by this via the working electrode 10. Thus, the measurement target substance 6 is detected.

Note that if the redox product 8 is the same as the mediator 14, this product can participate in the enzyme reaction again.

When the measuring cell 101 of the detector 100 includes plural electrodes, water may be generated on any of these electrodes, e.g., on an electrode paired with the detection electrode 10. This reaction on the electrode is one of reactions pertaining to self-formation of water.

This water can return to the reaction field of the enzyme body 3. For example, the enzyme body 3 may include a reversed micelle including a water pool 4. In this case, at least a part of water generated by the reaction on the electrode enters the water pool 4 in the reversed micelle. Water generated on the electrode can enter the water pool 4 until the limiting amount of solubilized water of the reversed micelle is reached.

When the medium 2 of the mixture 102 includes an ionic liquid, excess water is discharged from the mixture 102 to the outside if the water amount in the water pool 4 reaches the limiting amount of solubilized water of the reversed micelle. Since the specific gravity of ionic liquid is larger than that of water, water moves above the ionic liquid. Phase separation thus occurs. Since the water phase is positioned above the ionic liquid phase, excess water is removed by evaporation.

As described above, it is possible to adopt an arrangement in which water generated by the reaction on the electrode is replenished to the water pool 4 of the enzyme body 3. In this arrangement, water in the water pool 4 is hardly depleted, so the enzyme 5 always shows high activity. Also, in the case that the enzyme reaction is hydrolysis, the hydrolysis of substrate is not prevented by a lack of water.

In the above-described example, the method of detecting the measurement target substance 6 by detecting the change in electrochemical property of the mixture 102 by electrochemical measurement has been explained. However, the method of detecting the measurement target substance 6 using the measuring cell 101 and detector 100 of the embodiment is not limited to electrochemical measurement method. For example, detection by an optical measurement method may be performed by using, as the measuring unit 9, a device such as a spectrophotometer capable of measuring optical properties. In addition, the detector 100 may also be a voltage sensor.

The measuring cell 101 as described above may be used even when detecting the measurement target substance 6 by measuring a change in optical property of the mixture 102. When using the optical measurement method, however, electrodes such as the detection electrode 10 and comparison electrode 11 may be omitted. Furthermore, the detector 100 may include plural measuring units 9 which perform measurements by different methods, and the measuring units 9 may perform measurements on a single measuring cell 101. In such a detector 100, for example both of detection of the measurement target substance 6 by electrochemical measurement, and detection of the measurement target substance 6 by optical measurement, can be performed on the same measuring cell 101.

The change in optical property of the mixture 102 may be measured by, e.g., measuring a change in absorbance of the mixture 102 at a specific wavelength. For example, the concentration of the product 7a of the enzyme reaction catalyzed by the enzyme 5 may be calculated by the Lambert-Beer law or the like by measuring the absorbance of the mixture 102 at a wavelength at which the absorption coefficient of the product 7a is known. Thus, the measurement target substance 6 can be detected by detecting the product 7a by optical measurement.

When using the Lambert-Beer law, the portion of the main cell member 1 of the measuring cell 101, which holds the mixture 102, desirably has a consistent thickness.

The mixture 102 may include a dye as needed. For example, a dye may be used as the mediator 14. Alternatively, an enzyme reaction which produces a dye as the product 7a may be used. In the case that a dye is used as the mediator 14, the concentration of the dye reduces due to the enzyme reaction, and thereby the absorbance of the mixture 102 reduces. In the case that a dye is produced by the enzyme reaction, the concentration of the dye increases, and thereby the absorbance of the mixture 102 increases. In either case, the measurement target substance 6 can be detected by detecting a change in optical property of the mixture 102, e.g., a change in absorbance.

It is also possible to detect the measurement target substance 6 by capturing an image of the mixture 102, and analyzing a color change of the mixture 102 caused by the enzyme reaction from the captured image based on colorimetric analysis. An apparatus to be used to capture an image of the mixture 102 is not particularly limited. For example, even a portable camera is satisfactory.

In the detector 100 using optical measurement, any optical measurement device may be used as the measuring unit 9 as long as the device can measure the optical property such as the absorbance or chromaticity of a sample. When the measuring cell 101 is detachable from the detector 100, the measuring cell 101 is attached to the detector 100 in a manner such that the optical property of the mixture 102 in the main cell member 1 can be measured using the measuring unit 9.

As has been explained above, by using the detector according to the first embodiment, a sample to be measured can be selectively detected at high sensitivity without using any aqueous electrolyte.

### Second Embodiment

A measuring cell according to the second embodiment has the same arrangement as that of the measuring cell according to the first embodiment, except that a mixture itself supported by or held in a main cell member includes a substrate. In the second embodiment, a measurement target substance included in a sample to be measured is an inhibitor for an enzyme included in an enzyme body.

FIG. 4 is a schematic view of an example of a detector according to the second embodiment.

As shown in FIG. 4, a detector 200 according to the second embodiment has the same arrangement as that of the detector 100 according to the first embodiment, except that a mixture 202 includes a substrate 15 in addition to a medium 2 and enzyme body 3.

The substrate 15 may exist in a supersaturation state in the mixture 202. In the mixture 202, a solid substrate 15, e.g., a powder of the substrate 15 is preferably dispersed in the medium 2.

In the second embodiment, a measurement target substance 6 is an inhibitor of an enzyme 5. Therefore, when the measurement target substance 6 is introduced to the mixture 202 including the enzyme body 3, an enzyme reaction in the enzyme body 3 is inhibited. As a consequence, the concentrations of products, e.g., products 7′a and 7′b formed by the enzyme reaction change. The detector 200 detects the measurement target substance 6, for example by detecting the concentration change of the product 7′a. When detecting the measurement target substance 6 in the second embodiment, the measurement target substance 6 may be detected by detecting the change in electrical property, electrochemical property, or optical property of the mixture 202, which is caused by the formation of the product 7′a, in the same manner as explained in the first embodiment.

When the product 7′a is an electrode active material, the electrochemical property change of the mixture 202 can be measured. The electrochemical property change can be measured by, e.g., the S1 measurement mode using only a detection electrode 10.

In the detector 200 shown in FIG. 4, it is also possible to measure the electrochemical characteristic change of the mixture 202 by the S2 measurement mode by using a pair of working electrodes, i.e., the detection electrode 10 and a comparison electrode 11.

In the detector 200 shown in FIG. 4, when the concentration of the measurement target substance 6 introduced to the mixture 202 in a measuring cell 201 increases, an enzyme reaction catalyzed by the enzyme 5 becomes more largely inhibited, and the formation of the product 7′a becomes more largely suppressed. The decrease in concentration of the product 7′a may be measured as a decrease in oxidation or reduction current value by the detection electrode 10.

Next, an inhibition rate (%) may be calculated based on the following equation (Equation 2), and the concentration of the measurement target substance 6 may be estimated based on the obtained inhibition rate.

Inhibition rate (%)=(|Iₜₙ−Iₜₙ₊₁|)/Iₜₙ×100 (Equation 2)

When performing quantitative measurement of the measurement target substance 6 based on the inhibition rate, the relationship between the inhibition rate and the concentration of the measurement target substance 6 may be confirmed beforehand. For example, a database constructed by forming a calibration curve may be stored in a data processor of a measuring unit 9.

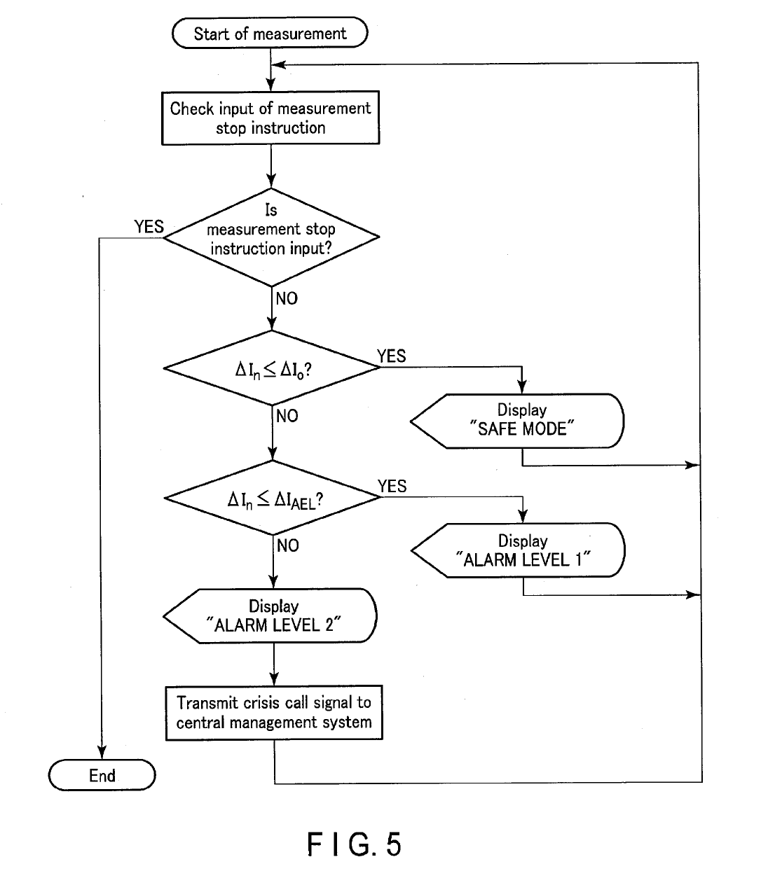

When the measurement target substance 6 included in a sample to be measured is a hazardous substance, the measuring unit 9 may also function, for example as an alarm having an alarm transmitting function. When the measuring unit 9 functions as an alarm, the measuring unit 9 can measure the measurement target substance 6 and transmit alarm in accordance with, e.g., a flowchart of measurement by chronoamperometry (CA) shown in FIG. 5. In this flowchart shown in FIG. 5, ΔIₒ is a constant defined in advance as a noise-level current change value, and may be, e.g., the difference (ΔIₒ=|I_{tn′+1}−I_{tn′}|) between a current value at first time and a current value at second time in a steady state in the S1 measurement mode explained in the first embodiment. This constant may alternatively be the difference (ΔIₙ=|I₁−I₂|) between the current value of the detection electrode 10 and the current value of the comparison electrode 11 in the steady state in the S2 measurement mode.

Regardless of whether the measurement mode is the S1 measurement mode or S2 measurement mode, an appropriate alarm signal can be generated based on the value of ΔIₙ.

For example, when ΔIₙ≦ΔIₒ, i.e., when ΔIₙ calculated by ΔIₙ=|Iₜₙ₊₁−Iₜₙ| or ΔIₙ=|I₁−I₂| is less than or equal to ΔIₒ derived from current noise, the concentration of the measurement target substance 6 which is, e.g., a hazardous substance may be determined to be lower than a detection level. In this case, the detector 200 may be operated in, e.g., a safe mode. In this safe mode, for example “SAFE MODE” may be displayed on a display panel or the like in accordance with an instruction by the measuring unit 9. In the safe mode, measuring of the measurement target substance 6 may be repeated.

When ΔIₙ>ΔIₒ, i.e., when ΔIₙ calculated by ΔIₙ=|Iₜₙ₊₁−Iₜₙ is greater than ΔIₒ derived from current noise and less than or equal to a current change value ΔI_{AEL} (ΔIₙ≦ΔI_{AEL}) corresponding to an acceptable exposure limit (AEL) of the measurement target substance 6, the detected concentration of the measurement target substance 6 may be determined to correspond to, e.g., alarm level 1. In this case, for example the measuring unit 9 may signal an alarm of alarm level 1. Signaling of the alarm of alarm level 1 may be performed by, e.g., displaying “ALARM LEVEL 1” on the display panel or the like. Alternatively, an alarm-indicating sound may be emitted using a buzzer or the like. After signaling the alarm of alarm level 1 or while continuously signaling the alarm, the measuring unit 9 may repeat measuring of the measurement target substance 6.

Note that in the repetitive measurement after the alarm of alarm level 1 is signaled, as Iₜₙ in ΔIₙ=|Iₜₙ₊₁−Iₜₙ|, Iₜₙ at time (tₙ) at which it has been determined that ΔIₙ≦ΔIₒ for the last time, i.e., Iₜₙ during safe mode may be used. Iₜₙ₊₁ may be a current value measured in the repetitive measurement.

When ΔIₙ>ΔIₒ, i.e., when ΔIₙ calculated by ΔIₙ=|Iₜₙ₊₁−Iₜₙ| is greater than ΔIₒ derived from current noise and furthermore, greater than the current change value ΔI_{AEL} (ΔIₙ>ΔI_{AEL}) corresponding to the acceptable exposure limit (AEL) of the measurement target substance 6, the detected concentration of the measurement target substance 6 may be determined to correspond to, e.g., alarm level 2. In this case, for example the measuring unit 9 may signal an alarm of alarm level 2. Signaling of the alarm of alarm level 2 may be performed by, e.g., displaying “ALARM LEVEL 2” on the display panel or the like. Alternatively, an alarm-indicating sound may be emitted using a buzzer or the like. After signaling the alarm of alarm level 2, the measuring unit 9 may transmit a crisis notification signal to, e.g., a central management system. The central management system having received the crisis notification signal may further execute measures against the hazardous substance by, e.g., transmitting an evacuation call signal and crisis measure signal across a network. After that, measurement may be interrupted or repeated without interrupting the measurement. Furthermore, in such a case, the alarm may be continuously signaled. When interrupting the measurement, for example a measurement stop instruction or the like may be input.

The central management system may exist outside the detector 200. The detector 200 may, for example wirelessly communicate with the external central management system. The detector 200 may be setup to automatically activate and execute a mode of performing transmission and communication to the central management system.

The main difference between the measuring cell 201 and the detector 200 including the measuring cell 201 according to the second embodiment from the measuring cell 101 and the detector 100 including the measuring cell 101 according to the first embodiment lies in the role of the measurement target substance 6 in the enzyme reaction in the enzyme body 3. The measurement target substance 6 itself is the substrate of the enzyme reaction in the first embodiment, whereas the measurement target substance 6 is an inhibitor of the enzyme 5 in the second embodiment. Except for this point and the point that in accordance to the former point, materials selectable as a substance which participates in the enzyme reaction of the enzyme 5 or the like are different, there is no practical difference between the first and second embodiments. Accordingly, all changes in design and the like applicable to the measuring cell 101 and detector 100 according to the first embodiment are applicable to the measuring cell 201 and detector 200 according to the second embodiment.

As has been explained above, by using the detector according to the second embodiment, a sample to be measured can be selectively detected at high sensitivity without using any aqueous electrolyte.

Members of the measuring cells and detectors of the embodiments will be described in detail below.

### 1. Main Cell Member

The measuring cell includes a main cell member 1. The main cell member 1 supports or holds the mixture including the medium 2 and enzyme body 3.

The main cell member 1 may be made of, e.g., an insulating material. Also, the main cell member 1 may be physically connected to the measuring unit 9, or may be wirelessly connected to the measuring unit 9. Furthermore, the main cell member 1 may also be detachable from the measuring unit 9.

The shape of the main cell member 1 is not particularly limited and may be, for example a vessel including a bottom surface having a shape such as a circle, square, rectangle, or ellipse. The mixture of the medium 2 and enzyme body 3 may be held in such a vessel-like main cell member 1 of a form of such a vessel. The shape of the main cell member 1 may be a plate including a surface having a shape such as a circle, square, rectangle, or ellipse. The mixture of the medium 2 and enzyme body 3 may be supported by such a plate-like main cell member 1.

The main cell member 1 may completely surround the portion housing the mixture as long as the measurement target substance 6 can be introduced to the mixture. Alternatively, the mixture may be exposed.

Furthermore, the main cell member 1 may be designed so as to form a space adjacent to the mixture. When forming a space like this, a portion surrounding the space is desirably made of an insulating material. An opening may be formed in this portion surrounding the space, as a path for introducing a sample to be measured including the measurement target substance 6. Furthermore, when a sample to be measured is, e.g., a solid sample, the material around the opening may be a material having high adhesion to the sample to be measured. For example, when the measurement target substance 6 is a volatile substance, the main cell member 1 may be pressed against the sample to be measured so as to close the opening by the solid surface of the sample to be measured. By doing so, the space adjacent to the mixture becomes a closed space including the sample to be measured as a part of the outer wall, and thus, the measurement target substance 6 can be efficiently sampled. Also, no pretreatment needs to be performed on a sample to be measured as described above, and this facilitates detection and measurement of the measurement target substance 6.

Note that when sampling the measurement target substance 6, packing material (filler, loading material), porous film, or spacer having a predetermined porosity may be disposed in the space adjacent to the mixture, in order to prevent contact between the mixture and the sample to be measured.

The volatile measurement target substance 6 which can be sampled as described above includes, e.g., the following substances. Acetaldehyde, which is a metabolite of alcohol, and formaldehyde, which is a carcinogen, can be sampled as the volatile measurement target substance 6 from a human body. These substances can be sampled, for example by directly pressing the opening of the main cell member 1 against the skin surface of a human body. An agricultural chemical remaining in crop can be sampled from the crop as a sample to be measured. Residual agricultural chemicals such as dichlorvos, parathion, and carbaryl can be continually detected by adhering the main cell member 1 on a crop. Freshness of food can be evaluated in a similar manner. Furthermore, it is possible to evaluate not only crop itself but also, e.g., components included in the soil of farmland. In addition, formaldehyde as the measurement target substance 6 can be sampled from building materials, which use timbers, paints, or adhesives, as samples to be measured.

When the measurement target substance 6 is a nonvolatile substance, for example a liquid sample to be measured may be put in the space adjacent to the mixture of the medium 2 and enzyme body 3. When the sample to be measured including the measurement target substance 6 comes in contact with the mixture, the measurement target substance 6 in the sample to be measured is selectively extracted to the mixture by liquid-liquid extraction and concentrated. As such, the measurement target substance 6 can be detected at high sensitivity. In addition, no pretreatment needs to be performed on the sample to be measured as described, and this facilitates detection and measurement of the measurement target substance 6.

The sample to be measured including the nonvolatile measurement target substance 6 which can be sampled as described above includes, e.g., the following substances. When using the measuring cell and detector of the embodiment for medical applications and health management, blood, saliva, tear, urine, and the like may be used as the sample to be measured. Also, the sample to be measured need not be a liquid. For example, it is possible to blow human breath into a space formed in the main cell member 1, and detect alcohol or acetone gas, which is a kind of biomarker gas, included in the breath as the measurement target substance 6. Furthermore, a pollutant included in polluted water as the sample to be measured can also be detected as the measurement target substance 6.

The measurement target substance 6 that can be detected and measured by the measuring cell and detector of the embodiment is not limited to the abovementioned substances, and the sample to be measured is not limited to those mentioned above. Also, the utilization form of the measuring cell and detector of the embodiment is not limited to the aforementioned forms, as long as the measurement target substance 6 can be introduced to the mixture of the medium 2 and enzyme body 3.

### 2. Medium

The mixture supported by or held in the main cell member 1 of the measuring cell includes the medium 2. The medium 2 includes a nonaqueous solvent. In the case that an electrode is disposed in the main cell member 1, and the electrical property or electrochemical property of the mixture in the main cell member 1 may be measured using the electrode, the medium 2 functions as an electrolyte solution.

It is desirable that the medium 2 as an electrolyte solution is nonaqueous electrolyte solution. In an aqueous electrolyte solution, evaporation of water and deposition of electrolyte may occur during long-term measurement. This may make it difficult to accurately measure the concentration of the measurement target substance 6 over a long period of time. When using an aqueous electrolyte solution, therefore, the lifetimes of the measuring cell and detector shorten, and may make quantitative measurement of the measurement target substance 6 difficult.

For the sake of safety, soybean oil, olive oil, paraffin, or an ionic liquid is desirable as the nonaqueous solvent used in the medium 2 of the embodiment. It is particularly desirable to use an ionic liquid as the nonaqueous solvent included in the medium 2 of the embodiment. When using an ionic liquid, the ionic liquid itself functions as an electrolyte, so it is unnecessary to dissolve another electrolyte. That is, concentration adjustment of an electrolyte is unnecessary. Furthermore, an ionic liquid has a potential window far wider than that of an aqueous solvent, and also has excellent electrical conductivity. Other advantages of an ionic liquid are low volatility and low flammability.

Various kinds of ionic liquids exist, and a new ionic liquid may also be synthesized as needed. Ionic liquids are classified into an aprotic ionic liquid (AIL) and protic ionic liquid (PIL), and they may be selectively used as needed. A mixture of AIL and PIL may also be used.

As the ionic liquid, e.g., 1-octyl-3-methylimidazolium bis(trifluoromethanesulfonyl)amide, [C₈mIm⁺][TFSA⁻] (TFSA⁻=(CF₃SO₂)N⁻, 1-alkylimidazolium bis(trifluoromethanesulfonyl)amide, [CₙImH⁺][TFSA⁻] (n=4 and 8), a room temperature ionic liquid (RTIL), triethyl sulfonium bis(trifluoromethyl sulfonyl)imide (TSBTSI), 1-butyl-3-methylimidazolium hexafluorophosphate ([bmim][PF₆]), 1-butyl-2,3-dimethylimidazolium bis(trifluoromethylsulfonyl)imide ([bmim][Tf₂N]), 1-ethyl-3-methylimidazolium bis(trifluoromethylsulfonyl)imide ([emim][Tf₂N]), octyl-3-methylimidazoliumhexafluorophosphate ([omim][PF₆]), 1-decyl-3-methylimidazolium bis(trifluoro-methylsulfonyl)imide ([dmim][Tf₂N]), 1-butyl-3-methylimidazolium tetrafluoroborate ([bmim][BF₄]), 1-dodecyl-3-methylimidazolium chloride [dmim][Cl], 1-methyl-3-octylimidazolium chloride (MOImCl), an ionic liquid [C₂mim][NTf₂], 1-butyl-3-methylimidazolium bis[(trifluoromethyl)sulfonyl]imide, [C₄mim][NTf₂], IL [C₈mim][Tf₂N] (1-octyl-3-methylimidazolium bis(trifluoromethylsulfonyl)amide), IL 2(1-ethyl-3-methylimidazolium bromide, emimBr), 1-ethyl-3-methylimidazolium trifluoromethanesulfonate ([emim][Tf]), 1-ethyl-3-methylimidazolium tetrafluoroborate ([emim][BF₄]), 1-butyl-3-methylpyridinium tetrafluoroborate ([bmpyri]BF₄), 1-butyl-3-methylpyrrolidinium tetrafluoroborate ([bmpyrro]BF₄), [bmim]BF₄, and 1-ethyl-3-methylimidazolium chloride ([emim][Cl]) may be used.

### 3. Enzyme Body

The enzyme body 3 includes one or more enzymes 5. The enzyme body 3 may be a single enzyme 5. Alternatively, the enzyme body 3 includes an immobilized enzyme 5. Enzyme immobilization herein mentioned includes bonding an enzyme to a support by a support bonding method, entrapping an enzyme in a polymer gel or microcapsule by an entrapping method, and bonding enzymes to one another by a crosslinking method. The enzyme body 3 obtained by immobilizing the enzyme 5 includes, e.g., a composite including a molecular aggregate formed by a dispersant and the enzyme 5, a microcapsule encapsulating the enzyme 5, and a composite including a support formed by a polymeric material or the like and the enzyme 5 supported on or included in the support. A biological cell or microorganism including the enzyme 5 may also be used as the enzyme body 3.

An enzyme reaction requires water in most cases. This is so because an enzyme is originally a biocatalyst which functions in water. An enzyme normally shows a high enzyme activity in water because the enzyme becomes flexible in water. By contrast, the activity of an enzyme significantly decreases in a waterless system. Also, when an enzyme reaction is, for example hydrolysis, water itself participates in the reaction as a reactive species.

The enzyme body 3 may include water, and this water can function as an enzyme reaction field of the enzyme 5. Therefore, the enzyme 5 shows a high enzyme activity in the enzyme body 3.

The enzyme bodies 3 form a mixture when dispersed in the medium 2 including a nonaqueous solvent.

In the measuring cell and detector according to the embodiment, optionally, the mixture may include one type of enzyme bodies 3 where each enzyme body 3 includes two or more kinds of enzymes 5. Alternatively, the mixture may include plural types of enzyme bodies 3 each including different kinds of enzymes 5. In this case, each enzyme body 3 may include only one kind of enzyme 5, or may include two or more kinds of enzymes 5.

When the mixture includes plural types of enzyme bodies 3 including different kinds of enzymes 5, a part of a product formed by an enzyme reaction in one enzyme body 3 may function as a substrate of an enzyme reaction in another enzyme body 3. Chemical substances are rapidly exchanged between individual enzyme bodies 3 included within the same system. Therefore, the product formed by the enzyme reaction in one enzyme body 3 rapidly moves to another enzyme body 3 and participates in the enzyme reaction there as a substrate.

Also, when the mixture includes plural types of enzyme bodies 3 including different kinds of enzymes 5, a product of an enzyme reaction in one enzyme body 3 may include water, while an enzyme reaction in another enzyme body 3 requires water as a reactive species. In this case, the water produced in one enzyme body 3 rapidly moves to the other enzyme body 3 and can be used in the enzyme reaction there.

### 3-1. Enzyme

As the enzyme 5 to be included in the enzyme body 3, it is possible to use, e.g., oxidoreductase, modified enzyme, hydrolase, synthase, transferase, eliminated enzyme, protein crosslinking enzyme, mutated enzyme, isomerase, crosslinking enzyme, antibody enzyme, lyase, ligase, and crystallized enzyme. Examples of types of these enzymes will be presented below, but the enzyme 5 which may be included in the enzyme body 3 is not limited to these examples.

For example, enzymes such as parathion hydrolase, organophosphorus hydrolase enzyme (OPH), cholinesterase (ChE), choline oxidase (ChO), butyrylcholinesterase (BChE), β-galactosidase, peroxidase (HRP), acetylcholinesterase (AChE), formaldehyde dehydrogenase, cholesterol esterase (ChEt), cholesterol oxidase (ChOx), glucose isomerase, glucose-1-oxidase, glucose oxidase, glucose dehydrogenase, glucose-6-phosphate dehydrogenase, inpertase, penicillinase, β-glucosidase, decarboxylase, ammonia lyase, monoamine oxidase, alcohol dehydrogenase (ADH), ascorbate oxidase, amino acid oxidase, alcohol oxidase, pyruvate oxidase, creatinase, adenosine deaminase, acyl-CoA oxidase, acyl-CoA synthetase, aspartate aminotransferase, aspartate β-decarboxylase, aspartase, acetate kinase, aminoacylase, aminopeptidase, amylase, alanine dehydrogenase, arabanase, arabinosidase, RNA polymerase, alkali xylanase, alkali cellulase, alkali protease, alkali lipase, aldehyde dehydrogenase, aldolase, α-acetolactate decarboxylase, α-chymotrypsin, isoamylase, isocitrate dehydrogenase, invertase, uricase, urease, urokinase, esterase, N-acetylneuraminate lyase, endo-β-glucanase, ω-hydroxylase, catalase, carboxylesterase, carboxypeptidase, carbonic anhydrase, γ-glutamine transpeptidase, xanthine oxidase, formate dehydrogenase, xylanase, xylan acetyl esterase, xylose isomerase, chymosin, guanosine-5′-phosphate synthetase, citrate synthetase, glycerol oxidase, glycerol kinase, glycerol-3-phosphate oxidase, glucoamylase, glucosyl transferase, glutamate decarboxydase, glutamate dehydrogenase, creatininase, creatinine deiminase, cretinase, chloroperoxidase, 5′-adenylate deaminase, colipase, cholesterol oxidase, thermolysin, sarcosine oxidase, sarcosine dehydrogenase, 3-α-hydroxy steroid dehydrogenase, 3-chloro-D-alanine chloride lyase, diaphorase, cyanate aldolase, cyclodextrin glycosyl transferase, dihydropyrimidinase, streptokinase, superoxide dismutase, subtilisin, cephalosporin acylase, cephalosporin amidase, cellulase, cellobiohydrolase, cytochrome C, thymidylate synthase, DNA polymerase, deoxyribose-5-phosphate aldolase, dextranase, dopa decarboxylase, transglutaminase, triose phosphate isomerase, trypsin, tryptophanase, tryptophan synthetase, naringinase, nitrile hydratase, lactate dehydrogenase, neuraminidase, halohydrin epoxidase, halohydrin halogen halide lyase, haloperoxidase, histidine ammonia lyase, hydantoinase, pyranose-2-oxidase, phenyl alanine ammonia lyase, phenol oxidase, putrescin oxidase, flavoenzyme, purine nucleoside phosphorylase, pullulanase, protease, prourokinase, proteinase, proline iminopeptidase, bromoperoxidase, hexokinase, pectinase, pectin esterase, pectin transeliminase, β-etherase, β-glucanase, β-glucoamylase, β-fructofuranosidase, β-fractofuranosidase, peptidase, hemicellulase, penicillin amylase, penicillin amidase, pentosanase, phosphodiesterase, phospholipase, phosphorylase, polygaracturonase, mannnanase, mutanase, mutarotase, lactase, lactonohydrolase, lactoperoxidase, lactamase, racemase, laccase, lignin peroxidase, lysyl endopeptidase, lysine oxidase, lysine decarboxylase, lysozyme, lipase, ribulose-1,5-bisphosphate carboxylase, lipoprotein lipase, ribonuclease A, malate dehydrokinase, luciferase, leucine aminopeptidase, and rhodanase may be used. However, the enzyme 5 is not limited to these examples. An artificial enzyme newly created by gene recombination may also be used.

As the antibody enzyme, antibody enzymes having antigen specificity for antigens existing in, e.g., influenza virus, AIDS virus, helicobacter pylori, cytokine, and IgE may be used.

### 3-2. Dispersant

An emulsifying agent may be used as the dispersant. An emulsifying agent is an amphipathic molecule having a hydrophilic group and hydrophobic group. The kinds and combinations of emulsifying agents used in the embodiment are not particularly limited, as long as a stable molecular aggregate can be formed using the emulsifying agent. For example, a lipid, boundary lipid, sphingolipid, fluorescent lipid, a cationic surfactant, an anionic surfactant, an amphoteric surfactant, a nonionic surfactant, a synthetic polymer, and a natural polymer such as protein may be selected as appropriate, to be used as the emulsifying agent.

When using a lipid as an emulsifying agent, for example triolein, monoolein, egg yolk lecithin, phospholipids, synthetic lipids, lysophospholipids, glycosyl diacylglycerols, plasmalogens, sphingomyelins, gangliosides, fluorescent lipid, sphingolipid, glycosphingolipid, lecithin, steroid, sterols, cholesterol, cholesterol oxide, dihydro cholesterol, glyceryl distearate, glyceryl monooleate, glyceryl dioleate, isosorbate monobrassidate, sorbitan tristearate, sorbitan monooleate, sorbitan monopalmitoleate, sorbitan monolaurate, sorbitan monobrassidate, dodecylic acid phosphate, dioctadecyl phosphate, tocophenol, chlorophyll, xanthophyll, phosphatidylethanolamine, phosphatidylserine, inositol, hexadecyltrimethylammonium bromide, diglycosyl diglyceride, phosphatidylcholine, retinal/cholesterol oxide/lectin/rhodopsin, all brain lipids, and all human red cell lipids may be used.

When using various surfactants as the dispersant, for example surfactants such as alkyl quaternary ammonium salt (e.g., CTAB and TOMAC), alkyl pyridinium salt (e.g., CPC), dialkyl sulfosuccinate (e.g., AOT), dialkyl phosphate, alkyl sulfate (e.g., SDS), alkyl sulfonate, a polyoxyethylene-based surfactant (e.g., the surfactants of Tween®, Brij®, and Triton® series), alkyl sorbitan (e.g., the surfactants of Span® series), a lecithin-based surfactant, a pluronic-type nonion surfactant, a pluronic-type cation surfactant, a betaine-based surfactant, and sucrose fatty acid ester (sugar surfactants) may be used. Surfactant as the dispersant used in the embodiment is not limited to these examples.

When using an ionic liquid as the dispersant, for example a protonic ionic liquid such as 1-alkylimidazolium bis(trifluoromethanesulfonyl)amide, [CₙImH⁺][TFSA⁻] (n=4 and 8) may be used.

When using a polymer as the dispersant, for example polysorb, polyethylene glycol, polyvinyl alcohol, propylene glycol, and comb-like polyethylene glycol may be used.

When using protein as the dispersant, for example casein or the like may be used.

Pluronic may also be used as the dispersant.

### 3-3. Molecular Aggregate

In the medium 2, by using the dispersant, one or more molecular aggregate selected from a nearly spherical reversed micelle or reverse wormlike micelle, liposome, vesicle, a microemulsion, a larger emulsion, a bicontinuous microemulsion, a monodispersed single emulsion, a double emulsion, and a multilayered emulsion may be formed.

The enzyme body 3 may be obtained by immobilizing the enzyme 5 to such a molecular aggregate. As an example of the molecular aggregate, a nearly spherical reversed micelle formed in the medium 2 by the dispersant can maintain a considerable amount of water in a central portion as the water pool 4. The enzyme 5 may be immobilized by being entrapped in the water pool 4 of the reversed micelle. Such immobilization of the enzyme 5 is referred to as solubilization of the enzyme 5 to the water pool 4. In the enzyme body 3, the water pool 4 may be used as the field of the enzyme reaction catalyzed by the enzyme 5.

The reversed micelle may be formed, for example as follows. An emulsifying agent may be added to a nonaqueous solvent. When the concentration of the emulsifying agent reaches a critical micelle concentration (CMC), a hydrophilic group and hydrophobic group of the emulsifying agent respectively face the inside and outside, thereby forming a nearly spherical reversed micelle surrounding water.

By further increasing the concentration of the emulsifying agent and thereby growing the spherical reversed micelle, a reverse wormlike micelle can be formed. Water within the interior of the reverse wormlike micelle may be the reaction field of the enzyme reaction like that in the reversed micelle. Also, by using the reverse wormlike micelle as the enzyme body 3, a mixture including the medium 2 and enzyme body 3 can be gelled. Details of gelling the mixture will be described later.

Reversed micelles or reverse wormlike micelles may also be formed, for example by adding a surfactant such as AOT, instead of an emulsifying agent, to a nonaqueous solvent. Reverse wormlike micelles can be formed by increasing the concentration of AOT in the nonaqueous solvent. When the AOT concentration is further increased, the reverse wormlike micelles become intertwined, and the whole mixture becomes gelled.

As another molecular aggregate, for example liposome, vesicle, a microemulsion, a larger emulsion, a bicontinuous microemulsion, a monodispersed single emulsion such as a water-in-oil type emulsion (W/O monodispersed emulsion), a double emulsion (W/O/W double emulsion), and a multilayered emulsion, formed by the dispersant may be used. These molecular aggregates may include an internal water phase or water phase that may be used as the water pool 4.

In the water pool 4, water bounding to a dispersant caused by ion-dipole interactions, or existing in vicinity of a hydrophilic group of a protonic ionic liquid (PIL) is called bound water. On the other hand, water existing in the central portion of the water pool 4 is free water in almost the same state as that of bulk water. Exchange is rapidly performed between the free water and bound water. The amount of free water increases as a water content ωₒ increases. The water content ωₒ is obtained by the following equation.

ωₒ=[H₂O]/[S] (Equation 3)

Here, [H₂O] is the molar concentration of water, and [S] is the molar concentration of a dispersant (S).

Also, the radius (R_{w}) of the water pool is obtained by the following equation.

R_{w}=0.15ωₒ (Equation 4)

When using a protonic ionic liquid (PIL) as the ionic liquid, the PIL functions as a cosurfactant, and contributes to the formation of reversed micelles or a microemulsion (water-in-ionic liquid type; W/IL), as well. Therefore, it is necessary to take account of the amount of PIL used in the formation of reversed micelles or a microemulsion (W/IL). Generally, the water content ωₒ increases as the PIL amount increases when the concentration [S] of a surfactant is constant.

The size of the water pool 4 can be appropriately adjusted by properly adjusting the water content ωₒ.

The above-described molecular aggregate such as a reversed micelle, reverse wormlike micelle, liposome, vesicle, microemulsion, larger emulsion, W/O monodispersed emulsion, or W/O/W double emulsion may further be coated with a gel or polymeric material.

The molecular aggregate such as a reversed micelle, liposome, vesicle, microemulsion, larger emulsion, W/O monodispersed emulsion, or W/O/W double emulsion coated with a gel or polymer can be regarded as a microcapsule.

To increase the stability of the molecular aggregate, the efficiency of the enzyme reaction, or the efficiency of detection of the enzyme reaction product, one or more types of materials selected from graphene oxide, carbon nanotubes, graphene, carbon nanohorns, silica nanoparticles, silver nanoparticles, gold nanoparticles, palladium nanoparticles, semiconductor nanoparticles, and a mesoporous material may be dispersed in the interior, on the surface, or in the periphery of the molecular aggregate. The interior of the molecular aggregate is, e.g., the water pool of a reversed micelle or the interior of a reverse wormlike micelle. Of these materials, when graphene oxide, carbon nanotubes, graphene, carbon nanohorns, silver nanoparticles, gold nanoparticles, or palladium nanoparticles are dispersed, a high electron conductivity, a high ion conductivity, and an effect of improving the stability of the molecular aggregate can be obtained. On the other hand, when silica nanoparticles, semiconductor nanoparticles, or a mesoporous material is dispersed, the effect of improving the stability of the molecular aggregate can be obtained.

### 3-4. Microcapsule

The microcapsule according to the embodiment refers to, for example a capsule obtained by encapsulating a core including a micronucleus (solid, liquid, or gas) with a porous membrane, and having a size from a nanoscale to a millimeter scale. This microcapsule in the enzyme body 3 has effects of, e.g., modifying the enzyme 5, and isolating, saving, and hiding the enzyme 5 from the nonaqueous solvent.

The core of the microcapsule according to the embodiment may be used as the enzyme reaction field. In addition, the microcapsule can rapidly entrap, to the core, components which participate in the enzyme reaction, such as the measurement target substance 6, substrate 15, mediator 14, water, and product, and can also rapidly release the enzyme reaction product from the core.

As the membrane of the microcapsule, i.e., as the material of a shell, it is possible to use a hygroscopic polymeric material or another polymeric material that may be used as a support. That is, the membrane of the microcapsule may be one kind of an organic membrane made of a hygroscopic polymeric material or a polymeric material, an inorganic membrane, and an inorganic-organic hybrid membrane.

Generally, the microcapsule may be formed by the three major methods, i.e., the chemical method, physicochemical method, and mechanical/physical method. Of these methods, examples of a method of forming a spherical mononuclear microcapsule include interfacial polymerization, in-situ polymerization, and in-liquid cured coating method as chemical methods, and in-liquid drying as a physicochemical method.

The microcapsule according to the embodiment may be formed by the above-described methods, and may also be formed by using a double emulsion formed by, e.g., two-step emulsification, membrane emulsification, or one-step emulsification as a template. A microcapsule obtained using a double emulsion formed by one-step emulsification as a template is particularly desirable because the amount of impurities in the core substance is small, variation in the particle size, the number of cores, and the particle size of the core is small, and the enzyme can be encapsulated in the core while maintaining high activity.

The microcapsule may also be formed by photopolymerization of a reactive dispersant by using a reversed micelle, vesicle, or double emulsion formed by the dispersant.

The enzyme body 3 may be a microcapsule that has an enzyme 5 maintained therein. Such an enzyme body 3 can be obtained by forming a microcapsule so that the microcapsule encapsulates the enzyme 5, when forming the microcapsule by the above-described method. The microcapsule may also maintain a cell or microorganism (to be described below), instead of the enzyme 5, in the microcapsule. Before the microcapsules (enzyme bodies 3) obtained as described above and encapsulating the enzyme 5 are dispersed in the medium 2, the microcapsules may be immersed in an aqueous solvent such that the core or membrane includes water.

### 3-5. Cell and Microorganism

A biological cell or microorganism including the enzyme 5 may be used as the enzyme body 3. A cell or microorganism may singly be used as the enzyme body 3. It is also possible to use a cell or microorganism immobilized by support bonding or entrapping as the enzyme body 3.

The enzyme body 3 may also be a cell or microorganism coated with a gel or polymeric material. Details of the gel or polymeric material coating a cell or microorganism will be described later. When coating a cell or microorganism with a gel, extracellular matrix protein (ECM protein) or fibronectin (FN) as an extracellular matrix may also be used together with the gel to coat the cell or microorganism.

Cells and microorganisms existing in nature include various enzymes, and there exist cells and microorganisms having enzymes or combinations of enzymes useful for the measuring cell and detector of the embodiment. A cell or microorganism having an appropriate combination of enzymes may be selected to be used as the enzyme body 3 of the embodiment. Also, a cell that may be used for the embodiment may be a cell other than a microorganism, e.g., an animal cell or plant cell.

A cell or microorganism may be used in a dead state where no reproduction occurs. Note that a microorganism in this dead state is in a resting state. When this microorganism in the resting state is immobilized, it is referred to as an immobilized resting cell.

### 3-6. Support

As the support for immobilizing the enzyme, for example polysaccharides such as powder-like or porous bead-like chitin, chitosan (e.g., CHITO PEARL BCW3010® manufactured by FUJIBO), xylan, and K-carrageenan may be used. As the support, for example porous glass, polylactic acid, alumina, silica gel, and celite may be used, also. In addition, for example polysaccharide derivatives such as cellulose, dextran, and agarose may be used as the support. Cellulose may be used in the form of nonwoven fabric.

The abovementioned support may be modified by the enzyme 5 by a support bonding method (physical adsorption method, ionic bonding method, or covalent bonding method), or the enzyme 5 may be dispersed onto the support, thereby forming a composite. Alternatively, a 3D lattice-like structures of support, for example, may be modified with enzyme by an entrapment method (3D lattice-like structures type), and a composite may be formed by dispersing the enzyme within the network structure of the support. The composite obtained as such may be used as the enzyme body 3.

The support for immobilizing the enzyme 5 may be a hydrophilic or hygroscopic material. By using, e.g., a hygroscopic polymer as the support, water included in a sample to be measured or air can be collected to the support. By thus entrapping water into the enzyme body 3 from outside the main cell member 1, water necessary for the enzyme reaction can be supplied to the enzyme body 3.

As hygroscopic polymer (superabsorbent polymer) that may be used as the support, available are those made from a natural polymer or synthetic polymer.

A hygroscopic polymer made from a natural polymer is excellent in speed of water absorption. As the natural polymer, for example starch-based polymers (e.g., starch-acrylonitrile graft polymer hydrolysate, starch-acrylic acid graft polymer, starch-styrene sulfonic acid graft polymer, starch-vinyl sulfonic acid graft polymer, and starch-acrylamide graft polymer), cellulose-based polymers (e.g., a cellulose-acrylonitrile graft polymer, a cellulose-styrene sulfonic acid graft polymer, and a crosslinked carboxymethylcellulose), other polysaccharide-based polymers (hyaluronic acid and agarose), and protein-based polymers (e.g., collagen) may be used.

A hygroscopic polymer made from a synthetic polymer is excellent in mechanical strength and chemical stability. As the synthetic polymer, for example polyvinyl alcohol-based polymers (e.g., a polyvinyl alcohol crosslinked polymer and PVA water-absorbing gel, elastomer), acryl-based polymers (e.g., a crosslinked sodium polyacrylate, sodium acrylate-vinyl alcohol copolymer, and polyacrylonitrile-based polymer saponified product), other addition polymers (e.g., a maleic anhydride-based polymer and vinyl pyrrolidone-based copolymer), polyether-based polymers (e.g., a polyethyleneglycol-diacrylate crosslinked polymer), and condensation polymers (an ester-based polymer and amide-based polymer) may be used.

The above-described hygroscopic polymer may be processed into various forms such as a powder, bead, fiber, film, and nonwoven fabric in accordance with applications.

With the aforementioned hygroscopic polymer as a support, the support may be modified with enzyme by the support bonding method (physical adsorption method, ionic bonding method, or covalent bonding method), thereby dispersing the enzyme onto the support and forming the enzyme body 3. Alternatively, a 3D lattice-like structures of support, for example, may be modified with enzyme by the entrapment method (lattice type), or the enzyme may be dispersed in the network structure of the support, thereby forming the enzyme body 3.

A polymer gel may also be used as the support for immobilizing an enzyme. As this gel, for example Metrogel® (Metro Hydrogel®) made of a protein tropoelastin, gelatin methacrylate (GelMA) hydrogel, gelatin, alginate hydrogel, sodium polyacrylate gel, Mebiolgel® (manufactured by IKEDA KAGAKU), ambient temperature solidifying stretchable hydrogel AQUAJOINT® (manufactured by NISSAN CHEMICAL), silica gel, agar, κ-carrageenan, and polyacrylamide gel may be used.

The enzyme body 3 may be formed by dispersing an enzyme onto the abovementioned gel or modifying the gel with enzyme by the bonding method (physical adsorption method, ionic bonding method, or covalent bonding method), or encapsulating the enzyme by the gel by the entrapment method.

As the gel, a hydrogel, which includes water as a main solvent, may be used. Alternatively, an organogel, which includes a nonaqueous solvent as a main solvent, may be used.

When detecting the measurement target substance 6 by measuring the optical properties of the medium 2 and enzyme body 3, the support is desirably selected as not to hinder the translucency of the mixture. As such a support, for example a cellulose powder, cellulose nanofiber (CNF), cellulose nanocrystal (CNC), chitin nanofiber, or chitosan nanofiber may be used. A typical CNF has a width of about 4 to 100 nm and a length of about 5 μm, and a typical CNC has a width of about 10 to 50 nm and a length of about 100 to 500 nm. Also, for example [BiNFi-s], which is a nanofiber derived from cellulose, chitin, and chitosan manufactured by SUGINO MACHINE, may be used. [BiNFi-s] has a diameter of about 20 nm and a length of a few μm.

### 4. Mediator

The kind of the mediator 14 according to the embodiment is not particularly limited, provided that the mediator 14 is a substance which functions as a mediator of the enzyme reaction catalyzed by the enzyme 5.

When forming the enzyme body 3, the enzyme body 3 may be formed such that the mediator 14 is dispersed in the enzyme reaction field of the enzyme body 3 in advance. Alternatively, the mediator 14 such as oxygen may be supplied by breathing from the atmosphere to the enzyme reaction field of the enzyme body 3 through the mixture including the medium 2 and enzyme body 3.

The mediator 14 may also be dispersed in the mixture in the form of a powdery solid soluble in the medium 2 or water pool 4 such that the mediator 14 is supersaturated. The supersaturated mediator 14 dispersed in the mixture moves to the enzyme reaction field of the enzyme body 3 due to solid-liquid extraction, and participates in the enzyme reaction. When the mediator 14 is dispersed in the medium 2 in a supersaturation state, an advantage lies in that the mediator 14 can always be provided to the enzyme reaction field at a constant concentration.

Furthermore, a product formed by a reaction at an electrode, e.g., an oxidation-reduction reaction, can move back to the enzyme reaction field of the enzyme body 3, and may be used as the mediator 14.

Optionally, plural kinds of mediators 14 may be used in one measuring cell. When one or more enzyme bodies 3 include plural kinds of enzymes 5, different kinds of mediators 14 may be associated with different enzyme reactions. Alternatively, two or more different kinds of mediators 14 may be associated with the same enzyme reaction.

As the mediator 14, for example a ferrocene/ferricinium ion, potassium ferricyanide/potassium ferrocyanide, p-benzoquinone/hydroquinone, p-cresol, pyrogallol/purpurogallin, iodine, p-nitrophenol, phenol, aromatic amine, nicotinamide adenine dinucleotide (NADH) (reduced form)/nicotinamide adenine dinucleotide (NAD⁺) (oxidized form), and 3,3′,5,5′-tetramethylbenzidine (TMB)/3,3′,5,5′-tetramethylbenzidine diimine may be used.

### 5. Substrate

When the substrate is the measurement target substance 6 itself as in the first embodiment, the substrate need not be dispersed inside and outside the enzyme body 3 beforehand. On the other hand, when the measurement target substance 6 is not the substrate of the enzyme reaction as in the second embodiment, the substrate 15 may be dispersed in the enzyme reaction field of the enzyme body 3 beforehand.

Also, the substrate 15 may be dispersed in the medium 2 in the form of a powder-like solid soluble in the medium 2 or water pool 4 such that the substrate 15 is supersaturated, and move the substrate 15 to the enzyme reaction field of the enzyme body 3 by solid-liquid extraction. When the substrate 15 is dispersed in the medium 2 in a supersaturation state, the substrate 15 necessary for the enzyme reaction can be provided over a long period of time.

When the substrate 15 is not the measurement target substance 6, for example acetylthiocholine (ATCh), acetylcholine chloride (ACh), S-butyrylthiocholine chloride (BTChCl), choline (Ch), acetylthiocholine chloride (ATChCl), or acetylthiocholine perchlorate may be used as the substrate 15.

### 6. Mixture

The mixture includes the medium 2 and enzyme body 3. When the mediator 14 participates in the enzyme reaction of the enzyme body 3, the mixture may further include the mediator 14. Furthermore, in the second embodiment, the mixture further includes the substrate 15.

The mixture may be supported by or held in the main cell member 1.

The mixture may be held by the main cell member 1 by, e.g., being impregnated in a support. For example, the mixture may be held by impregnating the medium 2 including the enzyme body 3 into nonwoven fabric.

Optionally, the mixture may be gelled. For example, a mixture including the medium 2 including a nonaqueous solvent and the enzyme body 3 may be made into an organogel.

The mixture may be made into an organogel by, e.g., dispersing reverse wormlike micelles or nanofibers in the nonaqueous solvent included in the medium 2. Here, the reverse wormlike micelle or nanofiber may be a part of the enzyme body 3. The mixture may also be gelled by dispersing organic nanotubes having an inner diameter of about 10 nm in the nonaqueous solvent. Furthermore, the mixture may be gelled by crosslinking nonaqueous solvent molecules. When the enzyme body 3 includes a reversed micelle or reverse wormlike micelle, an organogel may also be formed by gelling the water pool 4 in the reversed micelle or reverse wormlike micelle by including gelatin or lecithin in the water pool 4.

Gelation of the mixture facilitates supporting the mixture on the main cell member 1. In addition, the gelled mixture has stability higher than that of a liquid mixture. For example, when the mixture is gelled, the distribution of the enzyme bodies 3 dispersed in the medium 2 is hardly biased due to the influence of, e.g., an impact from outside the main cell member 1.

The mixture may be made to be supported on the main cell member 1 by, e.g., coating an electrode such as the detection electrode 10 with the mixture by using a method such as ink-jet printing, dip coating, spin coating, spray coating, or casting. When coating the mixture, in a portion where no enzyme bodies 3 are dispersed in the medium 2 such as in vicinity of the comparison electrode 11, for example the comparison electrode 11 may be coated with only the medium 2. Alternatively, the comparison electrode 11 may be coated with a material in which the enzyme 5 is omitted from the enzyme body 3, e.g., the medium 2 including reversed micelles in which no enzyme 5 is solubilized into the water pool 4.

On the other hand, to abbreviate steps of coating the electrode with the mixture in order to reduce the cost, the detection electrode 10 and its counter electrode or a reference electrode may be coated with the same mixture.

After that, a gelled mixture may be obtained by gelling the mixture coated on the electrode.

### 7. Electrochemical Measurement

When detecting the measurement target substance 6 by measuring the change in electrical or electrochemical property of the mixture in the main cell member 1, one or more electrodes are disposed in contact with the mixture. Of the one or more electrodes, at least one is the detection electrode 10. As will be described later, the detection electrode 10 differs in its definition as an electrode depending on the method of measurement.

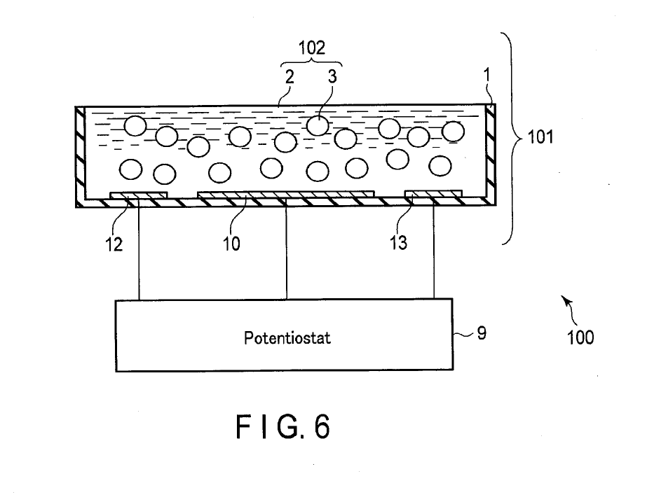

FIG. 6 shows a basic structure of the detector 100 for detecting the measurement target substance 6 by detecting, e.g., a product derived from the enzyme reaction of the substrate by an electrochemical measurement method (e.g., voltammetry).

In the detector 100 shown in FIG. 6, voltammetry, which is an electrochemical method, is used as the measurement method, and the measurement target substance 6 may be detected by measuring an oxidation-reduction reaction at the electrode using the above-described S1 measurement mode. In this detector 100, a working electrode of a potentiostat device is used as the detection electrode 10. The detector 100 shown in FIG. 6 also includes a reference electrode 12 and counter electrode 13 of the potentiostat device as electrodes.

The product 7a derived from the enzyme reaction in the enzyme body 3 may be measured by chronoamperometry. In this case, a voltage which is constant with respect to the reference electrode 12 may be applied to the detection electrode 10, and the potentiostat as the measuring unit 9 measures change in electric current with time (FIG. 2A). The measurement target substance 6 may be detected from calculation based on the behavior of change of the obtained electric current using the above-described method. Chronoamperometry is desirable when detecting for the presence of the measurement target substance 6 or measuring a change with time for the measurement target substance 6 over a long period of time, or when detecting the measurement target substance 6 in a flow system.

On the other hand, cyclic voltammetry may be used when measuring the measurement target substance 6 in a batch. From a current-potential curve obtained by cyclic voltammetry, a peak current value of oxidation or reduction of a product derived from an enzyme reaction may be obtained. The measurement target substance 6 may be measured based on the peak current value of oxidation or reduction of the electrode active material.

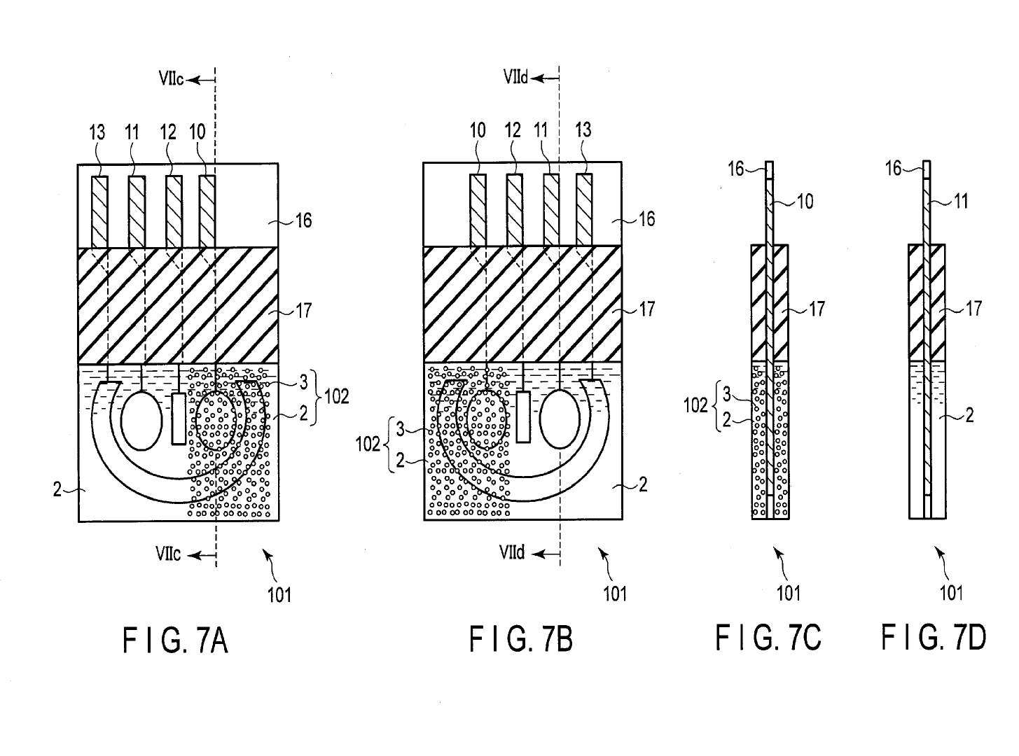

On the other hand, when measuring the electrode active material by the S2 measurement mode, the measuring cell 101 includes the comparison electrode 11 in addition to the detection electrode 10. FIGS. 7A, 7B, 7C, and 7D show an example of the measuring cell 101 using the S2 measurement mode in an electrochemical measurement method. In the measuring cell 101 shown in FIGS. 7A, 7B, 7C, and 7D, reverse faces of a printed electrode obtained by printing electrodes on reverse faces of a substrate 16 is further coated with the medium 2 or a mixture including the medium 2 and enzyme bodies 3. The measuring cell 101 further includes an electrical insulating layer 17. FIG. 7A schematically shows one face of the measuring cell 101, and FIG. 7B schematically shows the reverse face of the measuring cell 101. FIG. 7C is a sectional view of the measuring cell 101 taken along a broken line VIIc in FIG. 7A. FIG. 7D is a sectional view of the measuring cell 101 taken along a broken line VIId in FIG. 7B.

In the measuring cell 101 shown in FIGS. 7A, 7B, 7C, and 7D, both the detection electrode 10 and comparison electrode 11 are working electrodes, and the same reference electrode 12 and counter electrode 13 are shared. In the mixture 102 coating one face (e.g., the face shown in FIG. 7A) of the measuring cell 101, enzyme bodies 3 are dispersed near the detection electrode 10. Enzyme bodies 3 are also dispersed near the detection electrode 10 on the reverse face (e.g., the face shown in FIG. 7B) of the measuring cell 101. The kinds of the enzyme bodies 3 dispersed on one face of the measuring cell 101 and the enzyme bodies 3 dispersed on the reverse face may be the same or different. On the other hand, as shown in FIGS. 7A, 7B, 7C, and 7D, no enzyme bodies 3 are dispersed in vicinity of the comparison electrode 11 on either face of the measuring cell.

FIGS. 7A, 7B, 7C, and 7D show one working electrode as the detection electrode 10. However, plural working electrodes may be disposed as detection electrodes 10, and a single reference electrode 12 and single counter electrode 13 may be shared amongst the plural working electrodes (not shown).

Also, separate reference electrodes and counter electrodes may be used for each of the detection electrode 10 and comparison electrode 11. That is, the measuring cell 101 may include a first reference electrode and first counter electrode corresponding to the detection electrode 10, and a second reference electrode and second counter electrode corresponding to the comparison electrode 11 (not shown). In this case, in the medium 2 including a nonaqueous solvent, no enzyme bodies 3 are dispersed in vicinity of the comparison electrode 11 and second reference electrode.

By using the measuring cell 101 as described above and a bipotentiostat as the measuring unit 9, a product derived from an enzyme reaction may be measured with the S2 measurement mode. When performing electrochemical measurement by using chronoamperometry, a constant voltage (a voltage with respect to the reference electrode 12) may be applied to each of the detection electrode 10 and comparison electrode 11 in the measuring cell 101, and changes in electric currents with time for both electrodes may be measured by the bipotentiostat. If the measurement target substance 6 exists, a time change curve indicating the relationship between the electric current and time similar to that shown in FIG. 2B would be obtained.

In the detector 100 shown in FIG. 6 and the measuring cell 101 shown in FIGS. 7A, 7B, 7C, and 7D, a case is shown where a three-electrode electrochemical measurement method using the working electrode, reference electrode, and counter electrode is used; however, for example a two- or four-electrode electrochemical measurement method may also be used.

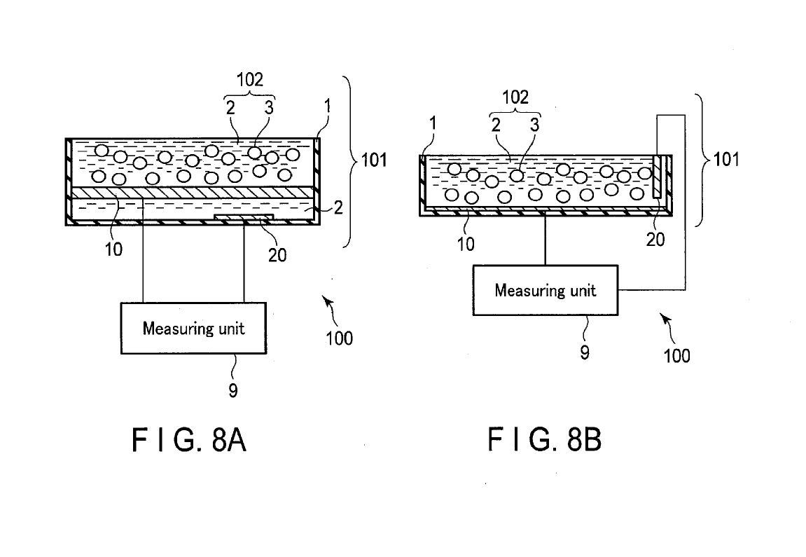

FIG. 8A schematically shows an example of the detector 100 using a two-electrode electrochemical measurement method. The detector 100 shown in FIG. 8A includes a mesh-like detection electrode 10 and an electrode 20 paired with the detection electrode 10. Only the mesh-like detection electrode 10 is in contact with the mixture 102 including the medium 2 and enzyme bodies 3. When an oxidation reaction occurs at the detection electrode 10, the detection electrode 10 is referred to as an anode. In this case, the electrode 20 paired with the detection electrode 10 is a cathode. On the other hand, when a reduction reaction occurs at the detection electrode 10, the detection electrode 10 is referred to as a cathode. In this case, the electrode 20 paired with the detection electrode 10 is an anode.

For example, a carbon cloth electrode, a graphene electrode having a porous structure, or the like may be used as the detection electrode 10.

As shown in FIG. 8B, the electrode 20 may also be disposed in contact with the mixture 102 including the medium 2 including a nonaqueous solvent and the enzyme bodies 3.

As the detection electrode 10, an electrode made of, e.g., platinum, gold, or titanium may be used. The electrode 20 paired with the detection electrode 10 may be selected in accordance with the measurement conditions, and for example, silver, platinum, palladium, or silver-silver chloride (Ag/AgCl) may be used.

Furthermore, a pseudo reference electrode may be used as the reference electrode 12. The pseudo reference electrode cannot sustain a constant potential. However, the potential of the pseudo reference electrode shows apparent dependence on measurement conditions. Therefore, since the potential can be calculated if the measurement conditions are known, the pseudo reference electrode may be used as the reference electrode 12.

As the reference electrode 12 and pseudo reference electrode, for example platinum, platinum black, palladium, silver, silver-silver chloride (Ag/AgCl), gold, or carbon may be used.

As the material composing the detection electrode 10 or comparison electrode 11, for example platinum, gold, or carbon, which is generally used from the viewpoints of chemical stability and reaction activity, may be used. Also, depending on the nonaqueous solvent included in the medium 2 the sample to be measured, for example a platinum-carbon electrode, gold-carbon electrode, tungsten electrode, titanium electrode, silver electrode, palladium electrode, graphene electrode, graphene oxide electrode, glassy carbon electrode, carbon cloth electrode, carbon paste electrode, semiconductor electrode (e.g., titanium dioxide), organic conductor, and diamond electrode may be used.

Furthermore, the detection electrode 10 or comparison electrode 11 may be processed into the form of, e.g., a flat plate, rod, mesh, wire, or cloth and used, in accordance with applications.

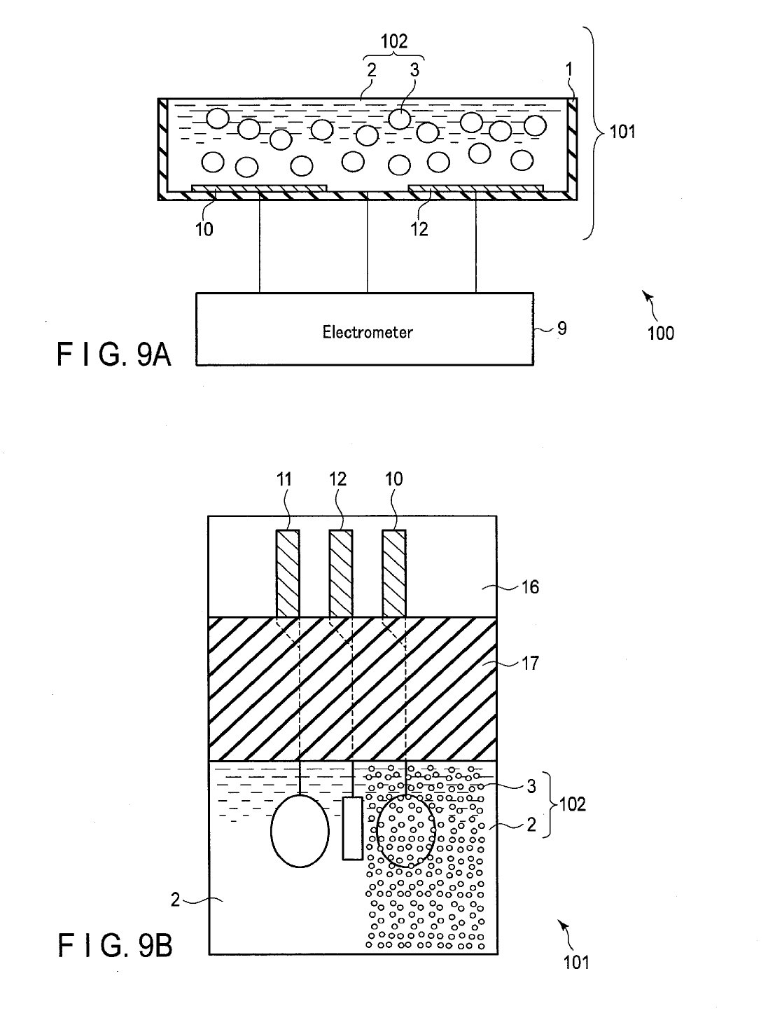

FIG. 9A schematically shows an example of the detector 100 using potentiometry as a measurement method and the S1 measurement mode. In this detector 100, for example an electrometer is used as the measuring unit 9, and an ion sensor of the electrometer is used as the detection electrode 10. The detector 100 also includes the reference electrode 12.

On the other hand, when using the S2 measurement mode, the measuring cell 101 and detector 100 further include a second ion sensor as the comparison electrode 11. FIG. 9B schematically shows an example of the measuring cell 101 using the S2 measurement mode by potentiometry. As shown in FIG. 9B, enzyme bodies 3 are dispersed in vicinity of the detection electrode 10 in the mixture 102. On the other hand, it is desirable that no enzymes 3 are dispersed in vicinity of the comparison electrode 11 and reference electrode 12 in the medium 2.

Although FIG. 9B shows one ion sensor as the detection electrode 10, plural ion sensors may also be disposed as the detection electrodes 10 in the same cell.

Furthermore, a different (second) reference electrode may also be disposed in a cell different from that of the detection electrode 10. In this case, an ion sensor disposed in the cell of the second reference electrode may be used as the comparison electrode 11. No enzyme bodies 3 are dispersed in the medium 2 in the cell in which the comparison electrode 11 and second reference electrode are disposed.

A change in mixture 102 derived from an enzyme reaction may be detected as a membrane potential by potentiometry. In a manner similar to the time change curve of an electric current shown in FIG. 2A, first, a change in membrane potential with time may be measured to obtain a time change curve indicating the relationship between the membrane potential and time. Then, quantitative measurement of the measurement target substance 6 may be performed based on the behavior of change of the membrane potential. When performing quantitative measurement of the measurement target substance 6, the relationship between the membrane potential and the concentration of the measurement target substance 6 may be confirmed in advance. For example, a database may be constructed based on the measurement in advance, and stored in a database processor of the measuring unit 9.

Optionally, the measuring unit 9 may be, e.g., a pH sensor for measuring hydrogen ions (pH). The measuring unit 9 may also be, e.g., an ammonium ion sensor for measuring ammonium ions. In this case, the reference electrode 12 need not be disposed in contact with the mixture 102. When disposing the detection electrode 10 and comparison electrode 11 in the same measuring cell 101, a single reference electrode 12 may be used for both the detection electrode 10 and comparison electrode 11.

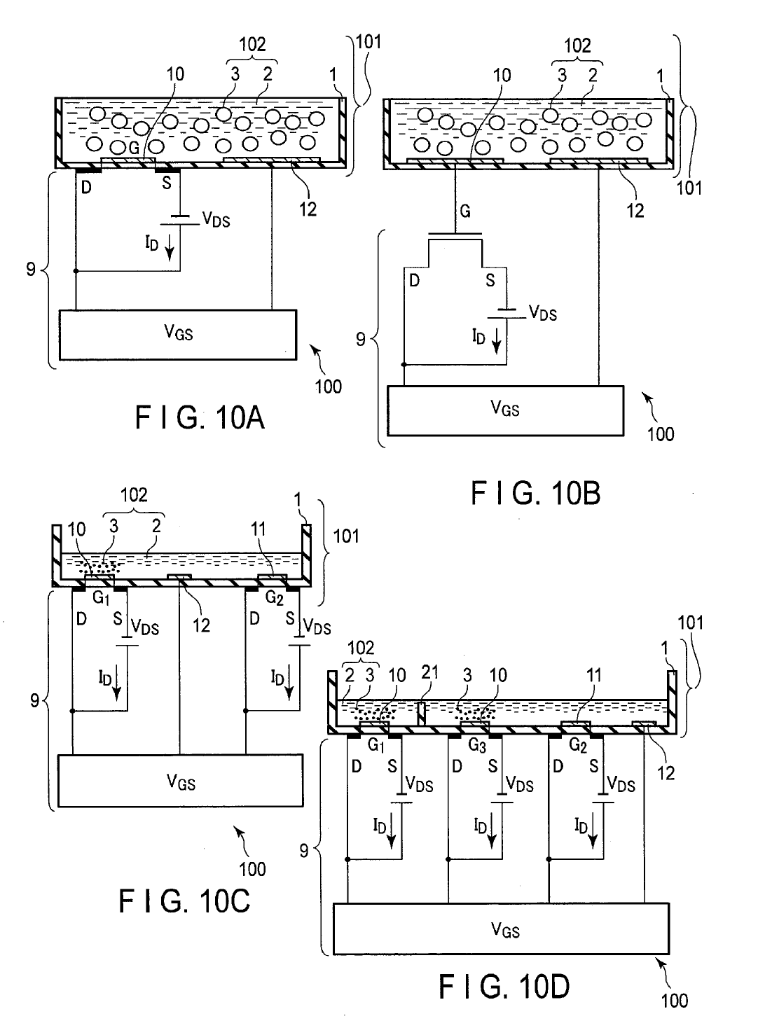

FIGS. 10A, 10C, and 10D each show the detector 100 including a field effect transistor (FET). FIG. 10B shows the detector 100 including an extended gate field effect transistor (EGFET). In the detector 100 including FET and EGFET, a gate electrode (G) is used as the detection electrode 10.

FIG. 10A shows the basic structure of the detector 100 including FET when using the S1 measurement mode. FIG. 10B shows the detector 100 including an extended gate field effect transistor (EGFET) when using the S1 measurement mode.

The detector 100 including FET detects a product of an enzyme reaction by using the modulation principle of a drain current caused by an interface potential change of the gate electrode (detection electrode 10).

In addition, a sensing portion (ion-sensitive film) capable of detecting a product of an enzyme reaction or a receptor molecule such as an antibody or aptamer may be formed on the gate electrode. This gives selectivity towards the measurement target substance 6 to be detected by the detector 100. Also, an ion selective field effect transistor (ISFET) can be obtained by disposing an ion selective film on the gate electrode. For example, an ion-sensitive film may be disposed on the gate electrode. A portion where the ion-sensitive film is disposed on the gate electrode is referred to as a sensing portion, hereinafter. A portion where no sensitive film is disposed is referred to as a gate electrode portion, hereinafter. In the case that at the sensing portion, an interaction between the sensing portion and a product of an enzyme reaction occurs, a change in potential of the gate electrode portion as a sensitive gate, i.e., a gate potential change is caused. Subsequently, a drain current is modulated due to the change in the gate potential of the gate electrode. Therefore, under the conditions where a voltage V_{DS} between a drain electrode (D) and source electrode (S) and a drain current I_{D} are constant, a change in interface potential of the gate electrode may be directly measured as a change in output voltage (V_{GS}) of a meter. When the relationship between the concentration of the product and the output voltage (V_{GS}) is confirmed in advance, the product may be quantitatively measured based on the relationship. The relationship between the product concentration and output voltage includes a calibration curve formed based on measurement in advanced, and may be stored as a database in the measuring unit 9.

FIG. 10C shows the basic structure of the detector 100 including FET when using the S2 measurement mode. FIG. 10D shows the detector 100 including a multichannel FET when using the S2 measurement mode.

As shown in FIG. 10C, a second gate electrode (G₂) as the comparison electrode 11 may be disposed in the same cell as that of a first gate electrode (G₁) as the detection electrode 10. In the detector 100 shown in FIG. 10C, the detection electrode 10 and comparison electrode 11 (G₁ and G₂) share the same reference electrode 12. In this case, as shown in FIG. 10C, no enzyme bodies 3 are dispersed in that portion of the medium 2 including a nonaqueous solvent, which is in contact with the comparison electrode 11 (G₂) and reference electrode 12. Such a detector 100 can measure a product of an enzyme reaction by the S2 measurement mode.

Furthermore, a multichannel FET may be obtained by disposing plural gate electrodes as the detection electrodes 10 in the same cell. As shown in FIG. 10D, of the three gate electrodes (G₁, G₂, and G₃), one gate electrode (G₂) may be used as the comparison electrode 11, and the two remaining gate electrodes (G₁ and G₃) may be used as the detection electrodes 10. In the mixture 102, the enzyme bodies 3 are dispersed in vicinity of the gate electrodes (G₁ and G₃) as the detection electrodes 10, and no enzyme bodies 3 are dispersed in vicinity of the gate electrode (G₂) as the comparison electrode 11 and the reference electrode 12. The types of the enzyme bodies 3 dispersed in vicinity of each of the gate electrodes (G₁ and G₃) as the detection electrodes 10 may be the same or different. When using different types of enzyme bodies 3, a partition 21 may optionally be disposed between the gate electrode (G₁) and gate electrode (G₃) as shown in FIG. 10D, in order to prevent the enzyme bodies 3 from diffusing and mixing with each other.

The detection electrode 10 and comparison electrode 11 may also be gate electrodes each disposed in different cells.

The detector 100 shown in FIG. 10D can simultaneously measure plural kinds of measurement target substances 6 by using plural gate electrodes as the detection electrodes 10.

Although each of the detectors 100 shown in FIGS. 10A, 10B, 100, and 10D includes the reference electrode 12, the reference electrode may be omitted.

When graphene is used as the material of the gate electrode, the detector is a graphene field effect transistor (GFET). In GFET, detection sensitivity can be increased to 10 to 1,000 or more as compared to a normal FET. Therefore, a detector including GFET is desirable.

Also, a graphene diode formed by n-G/G/p-G may be used. n-G is n-type graphene obtained by doping an n-type impurity such as nitrogen (N). p-G is p-type graphene obtained by doping a p-type impurity such as boron (B). G is graphene in which no impurity is doped. A graphene diode can be manufactured by joining p-G as a p-type semiconductor and n-G as an n-type semiconductor with graphene being interposed between them, and connecting the p-type semiconductor and n-type semiconductor to an external electric circuit. When using the graphene diode, the portion of graphene (G) may be used as the detection electrode 10.

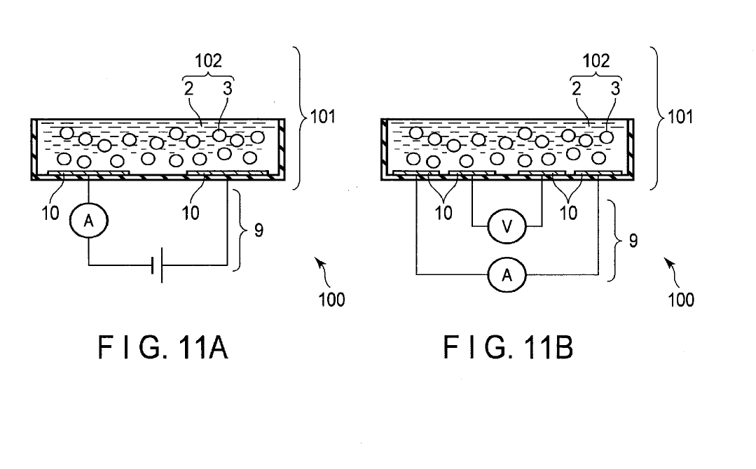

FIGS. 11A and 11B each show the basic structure of the detector 100 for detecting a sample to be measured by detecting the behavior of change in a product of an enzyme reaction as a conductivity or membrane resistance.

When detecting the behavior of change in a product of an enzyme reaction as a conductivity or membrane resistance by the S1 measurement mode, measurement may be performed by the following two measurement methods.

For example, the conductivity may be measured by a two-terminal method using the detector 100 as shown in FIG. 11A. When measuring the conductivity by the two-terminal method, a pair of electrodes is used as the detection electrodes 10 as a set.

An electric current may be supplied to the mixture 102 between the pair of electrodes, and the conductivity may be obtained by measuring a voltage drop of the mixture 102. The voltage measured by the two-terminal method includes results of voltage drops caused by various factors at the interface between the nonaqueous solvent included in the mixture 102 and the detection electrodes 10.

When measuring the conductivity, either a direct current or alternate current may be used. When taking account of the voltage drops in the interfaces, conductivity measurement by an alternate current is desirable. Conductivity measurement by a high-frequency alternate current is more desirable.

The conductivity may also be measured by a four-terminal method using, for example the detector 100 as shown in FIG. 11B. When measuring the conductivity by the four-terminal method, two pairs of electrodes, i.e., a pair of detection electrodes and a pair of current electrodes are used as the detection electrodes 10 as a set. Referring to FIG. 11B, the pair of detection electrodes are arranged on the inner side, and the pair of current electrodes are arranged on the outer side.

In the four-terminal method, an electric current may be supplied between the current electrodes on the outer side, and the conductivity may be obtained by measuring a potential difference between the detection electrodes on the inner side. A detector having a high internal resistance is desirably used to measure the potential difference between the detection electrodes on the inner side. Also, the measurement is desirably performed at a high frequency in order to avoid an error caused by the irreversibility of the current electrodes on the outer side.

In addition, the behavior of change in a product of an enzyme reaction may also be detected as the conductivity by using the S2 measurement mode.

Furthermore, the detector 100 may be structured as a graphene conductivity type sensor by using graphene as the detection electrode 10. The graphene conductivity type sensor is an electric resistance sensor, and uses a phenomenon in which the resistance of graphene changes when a molecule or ion as a detection target is adsorbed on the graphene surface as a sensing member. The graphene conductivity type sensor uses the principle that the carrier density and carrier mobility change when a molecule or ion is adsorbed by graphene.

When structuring the detector 100 as a graphene conductivity type sensor, either a graphene electrode or a graphene oxide electrode may be used as the detection electrode 10. The graphene electrode or graphene oxide electrode may be manufactured by, e.g., coating the surface of a carbon printed electrode with thin fragments of graphene or graphene oxide.

Details of the detector for detecting the measurement target substance 6 by measuring the change in electrical or electrochemical property of the mixture held in the main cell member 1 have been explained by taking the detector 100 according to the first embodiment as an example. These details are applicable not only to the detector 100 according to the first embodiment, but also to the detector 200 according to the second embodiment.

As the electrode, for example electrodes made of Pt, Au, Ag, carbon, graphene, graphene oxide, and a carbon nanotube coated on cellulose, paper, polymer nonwoven fabric, a thin porous film, and a thin polymer film, and a printed electrode printed on a substrate or the like may be used. A metal fiber may also be as an electrode. As the substrate for forming the printed electrode, a glass substrate, metal substrate, ceramics substrate, or polymer substrate may be used, but the kind of substrate is not particularly limited. Paper, nonwoven fabric, or a thin porous film may also be used as the substrate.

### 8. Optical Measurement

When detecting the measurement target substance 6 by measuring the change in optical property of the mixture accommodated in the main cell member 1, at least a part of the main cell member 1 is desirably made of a transparent material. Also, the mixture in the main cell member 1 is desirably adjusted so as to have transparency.

When a reactive species or product of the enzyme reaction in the enzyme body 3 is a substance which changes the optical property of the mixture, the measurement target substance 6 may be detected by optically measuring the substance. The mixture of the main cell member 1 may include a dye or the like as needed. The dye may be the mediator 14 which participates in the enzyme reaction, or a material which changes the optical property of the mixture by reacting with the reactive species or product of the enzyme reaction.

Examples of the dye that may be used in the measuring cell and detector of the embodiment include DCIP (2,6-dichlorophenolindophenol sodium salt), rhodamine B (RhB), chlorophyll, methylene blue, rose Bengal, cryptocyanine, and quinocyanine. In addition to the dye, a molecule having an absorption spectrum within a range from visible light to ultraviolet light or a fluorescent dye can achieve the same function as that of a dye molecule. Examples of the molecule having an absorption spectrum within the range from visible light to ultraviolet light include NADH, NAD⁺, pyrogellol, purpurogallin, and ferricyanide. An example of the fluorescent dye includes rhodamine 123.

The dye may be included in either the medium 2 or enzyme body 3 within the mixture.

### Third Embodiment

An analysis device according to the third embodiment includes the detector 100 according to the first embodiment or the detector 200 according to the second embodiment, and a sampling unit for vaporizing or ionizing the measurement target substance 6. The sampling unit of the analysis device of the embodiment includes at least one of a vaporizer for vaporizing the measurement target substance 6, and an ionization source for ionizing the measurement target substance 6.

The analysis device of the embodiment vaporizes or ionizes the measurement target substance 6 in the sampling unit, and then introduces the measurement target substance 6 to the measuring cell. The analysis device of the embodiment includes the sampling unit for vaporizing or ionizing the measurement target substance 6, and hence can efficiently sample the measurement target substance 6 from a sample to be measured. Thus the measurement target substance 6 can be detected and measured with higher precision. Also, since the sampling unit vaporizes or ionizes the measurement target substance 6, the measurement target substance 6 can rapidly be detected not only when a sample to be measured is a gas or liquid but also when it is a solid.

The measurement target substance 6 may be vaporized using, e.g., laser irradiation, UV irradiation, gas spraying, ultrasonic irradiation, heating, or voltage application. By vaporizing a solid or liquid sample using any of these methods, the sample can be sampled as a gas sample.

The measurement target substance 6 may be ionized using, for example a method of ionizing molecules using an ionization source. When ionizing molecules, the ionization method needs to be selected in accordance with conditions such as the molecular state, molecular weight, polarity, volatility, and molecular ionization energy of the measurement target substance 6. The molecular state is, for example whether the measurement target substance 6 is a solid, liquid, or gas.

Ionization methods can roughly be classified into a hard ionization method and soft ionization method.

In the hard ionization method, a fragmentation reaction of sample molecules is vigorous, and the sample molecules often thermally decompose or lose a functional group. In the hard ionization method, therefore, molecules are cut short at the same time as when they are ionized. In addition, in an ionization method classified as the hard ionization method, it is normally necessary to ionize molecules in a harsh environment such as a high vacuum.

In a typical method of ionizing molecules by the hard ionization method, molecules are ionized by, e.g., corona discharge, introduction of the molecules into a strong electrostatic field, or collision of thermions against the molecules.

The soft ionization method is a milder ionization method. The soft ionization method can generate gaseous ions while maintaining the molecular structure of a hardly volatile sample, and generation of fragment ions is little. Also, many ionization methods classified as the soft ionization method can ionize samples under atmospheric pressure, and require neither pretreatment nor separation of samples.

In a typical method of ionizing molecules by the soft ionization method, molecules are ionized by, e.g., an ionization reaction, a redox reaction, ion-attachment, or application of photon energy exceeding the ionization energy of the molecules of the sample.

As an ionization method conducted in the sampling unit using an ionization source, the soft ionization method is desirable because the method requires no pretreatment of a sample to be measured, generates few fragment ions, and does not require a special environment such as a vacuum environment, and hence, ionization of molecules for on-site analysis is possible. Of the soft ionization methods, the ambient ionization methods, such as paper spray ionization, desorption electrospray ionization, low temperature plasma probe (LTP), electrospray assisted laser desorption ionization, laser ablation electrospray ionization, and direct analysis in real time are further desirable.

In particular, low temperature plasma probe ionization (LTP) is favorable. LTP is an ionization method as a noninvasive noncontact sampling method. LTP can be used at a low temperature, consumes low electric power, and can use air as a discharge gas in a plasma source as an ionization source, and is therefore preferable. Also, using LTP, sampling of gas, liquid, and solid samples is possible. Therefore, when a nerve gas (a gas) as a chemical weapon agent or an explosive (a solid) is a measurement target substance, molecules of these substances can be ionized for on-site analysis, and thus, LTP can be used as an effective ionization method.

Furthermore, atmospheric pressure laser ionization (APLI) may also be used as the ionization method. In particular, use of a small-sized laser light source (a diode pumped solid state laser: DPSS) as an ionization source is preferable because a portable compact analysis device (analyzer) can be implemented.

In addition, when blood of a patient exposed to and poisoned by a chemical weapon agent is a sample to be measured and the chemical weapon agent included in the blood is to be sampled as the measurement target substance 6, for example paper spray ionization (PSI) may be used. PSI can directly ionize molecules of the measurement target substance 6 from the blood sample.

In the analysis device (analyzer) of the embodiment, the measurement target substance 6 included in a sample to be measured is vaporized or ionized in the sampling unit, and introduced to the measuring cell. The measuring unit measures and detects the vaporized or ionized measurement target substance 6, as has been explained for the detectors of the first and second embodiments. It is thus possible to analyze the sample to be measured and detect the measurement target substance 6.

In the sampling unit, the measurement target substance 6 included in the sample to be measured may be vaporized by the vaporizer. Alternatively, the measurement target substance 6 included in the sample to be measured may be directly ionized using the ionization source. Alternatively, the measurement target substance 6 may be vaporized by the vaporizer and then ionized using the ionization source.

As the vaporizer, for example a laser irradiator, UV irradiation, gas spray nozzle, ultrasonic irradiator, or heater may be used. A device that may be used as the vaporizer is not particularly limited as long as the device includes a means for vaporizing a sample.

When using, for example low temperature plasma probe ionization (LTP) as the ionization method, a plasma source may be used as the ionization source. When using, for example atmospheric pressure laser ionization (APLI) as the ionization source, a laser light source may be used as the ionization source. The ionization source is not particularly limited as long as the source can directly ionize the measurement target substance 6 or ionize a vaporized measurement target substance 6.

The analysis device of the embodiment can rapidly sample the measurement target substance 6 from a sample to be measured, because the abovementioned sampling unit vaporizes or ionizes the measurement target substance 6. Also, a sample to be measured including the measurement target substance 6 need only be placed in the sampling unit, and no special pretreatment for the sample to be measured is necessary, so analysis can be performed easily. Furthermore, since the analysis device includes detectors of the first and second embodiments, the analysis device is capable of highly selective sample analysis, and can be operated easily. In addition, reduction of cost and size of the analysis device can be accomplished easily because the detectors of the first and second embodiments have simple structures.

The analysis device of the embodiment can perform analysis on various samples to be measured, via noninvasive noncontact sampling of the measurement target substance 6. As an example of applications to agriculture, an agricultural chemical (dichlorvos) can be detected from fruits. Other agricultural chemicals such as parathion and carbaryl can be detected from samples to be measured such as fruits or vegetables and soil. As an application example other than agriculture, an explosive such as trinitrotoluene (TNT) can be detected.

A sample to be measured that can be analyzed and the measurement target substance 6 that can be detected by the analysis device are not limited to the aforementioned examples.

### EXAMPLES

Practical examples according to the first embodiment will be explained below.

### Example 1

A detector of Example 1 is a detector based on the first embodiment that is capable of detecting an agricultural chemical parathion.

The detector 100 of Example 1 includes one type of enzyme body 3 that includes one kind of enzyme 5. In the detector 100 of Example 1, the enzyme 5 is parathion hydrolase (PH), and catalyzes an enzyme reaction in which the substrate is parathion, which is also the measurement target substance 6.

In Example 1, a solution mixture of [C₈mIm⁺][TFSA⁻] as an aprotic ionic liquid (AIL) and [C₄ImH⁺][TFSA⁻] as a protic ionic liquid (PIL) is used as the medium 2=AIL/PIL=0.4). [C₈mIm⁺][TFSA⁻] is a hydrophobic ionic liquid, and [C₄ImH⁺][TFSA⁻] is a hydrophilic ionic liquid. [C₄ImH⁺][TFSA⁻] functions also as a cosurfactant.

Sodium 1,2-bis(2-ethylhexylcarbonyl)-1-ethane sulfonate (Aerosol OT: AOT) as an anionic surfactant is added to the solution mixture, and AOT (0.07 M) is dispersed by stirring the solution mixture for 20 hrs. Subsequently, a dilute buffer solution [0.02 M phosphoric acid/borate/acetate, pH=7] (0.02 M PBS) including parathion hydrolase (PH) as the enzyme 5 is added as an aqueous solution, and the solution mixture is stirred for 1 hr, thereby a reversed micelle or microemulsion (W/IL) made of AOT and [C₄ImH⁺][TFSA⁻], in which enzyme 5 is solubilized in a water pool 4, is prepared in the solution mixture of [C₈mIm⁺][TFSA⁻] and [C₄ImH⁺][TFSA⁻] as the medium 2.

By the abovementioned injection method, the reversed micelle or microemulsion (W/IL) in which PH is solubilized in the water pool 4 is formed as the enzyme body 3. The mixture 102 including this enzyme body 3 and the above-described medium 2 is thus obtained.

In the detector 100 of Example 1, when parathion as the measurement target substance 6 is introduced to the mixture 102, parathion enters the enzyme body 3 and is hydrolyzed by PH, thereby generating p-nitrophenol (PNP) (Reaction 1). This PNP may be detected by the S1 or S2 measurement mode using working electrodes (detection electrode 10 and comparison electrode 11) made of, e.g., platinum. The material for the working electrodes is not limited to platinum.

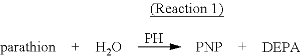

When performing the S1 measurement mode, for example a platinum electrode may be used as a counter electrode, and a platinum pseudo reference electrode may be used as a reference electrode. In the detector of Example 1, for example a potentiostat (Potentiostat/Galvanostat model 283 manufactured by EG & G) may be used as the measuring unit 9, and a constant potential within a range higher than the oxidation potential of PNP may be applied to the platinum working electrode (detection electrode 10). Thus parathion may be detected by measuring PNP, which is a hydrolysate of parathion.

When detecting parathion by performing the S2 measurement mode, for example platinum electrodes may be used as both the detection electrode 10 and comparison electrode 11. In this case, the enzyme bodies 3 are dispersed near the detection electrode 10 in the medium 2, but no enzyme bodies 3 are dispersed near the comparison electrode 11 in the medium 2. In the system of the main cell member 1, both of the two working electrodes, i.e., the detection electrode 10 and comparison electrode 11 are disposed in contact with the same mixture 102, so a single counter electrode and a single reference electrode may be shared by the two working electrodes. A constant potential (a potential with respect to the reference electrode) within the range higher than the oxidation potential of PNP may be applied to each working electrode.

Details of the S1 or S2 measurement mode are the same as those described above.

In the measuring cell 101 included in the detector 100 of Example 1, when the concentration of parathion introduced to the mixture 102 increases, an oxidation current of PNP also increases. A calibration curve indicating the relationship between the concentration and oxidation current of PNP may be prepared in advance, and this calibration curve may be stored as a database in a data processor of the measuring unit 9. By using the calibration curve, quantitative measurement of parathion may be performed based on the detected oxidation current value of PNP.

### Example 2

A detector of Example 2 is a detector based on the first embodiment that is capable of detecting an organic peroxide, e.g., 2-butanone peroxide.

The detector 100 of Example 2 includes one type of enzyme body 3 that includes one kind of enzyme 5. In Example 2, the enzyme 5 is peroxidase (HRP), and catalyzes an enzyme reaction in which the substrate is an organic peroxide (ROOH), which is also the measurement target substance 6. The detector 100 of Example 2 also uses ferrocene Fe(C₅H₅)₂ as the mediator 14 in the enzyme reaction in which an organic peroxide is the substrate.

The detector 100 of Example 2 has the same arrangement as that of the detector 100 of Example 1, except that the enzyme 5 is HRP, ferrocene is used as the mediator 14, and, when forming the mixture 102, a reversed micelle or microemulsion (W/IL) is formed by using, as an aqueous solution, a 0.05 M phosphoric acid buffer (0.05 M PBS, pH=7.4) including HRP as the enzyme 5.

In the detector 100 of Example 2, when an organic peroxide as the measurement target substance 6 is introduced to the mixture 102, the organic peroxide is reduced while ferrocene Fe(C₅H₅)₂ as the mediator 14 is oxidized into ferricinium ion [Fe(C₅H₅)₂]⁺ by the enzyme reaction catalyzed by HRP, which is the enzyme 5 of the enzyme body 3 (Reaction 2).

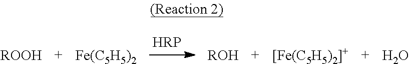

In the detector 100 of Example 2, in a similar manner as in Example 1, the ferricinium ion may be detected using working electrodes (detection electrode 10 and comparison electrode 11) made of, e.g., platinum, by applying a constant potential to the working electrodes and performing the S1 or S2 measurement mode. When performing the S1 measurement mode using the platinum working electrodes, a platinum electrode may be used as a counter electrode, and a platinum pseudo reference electrode may be used as a reference electrode. At the detection electrode 10, the ferricinium ion is reduced into ferrocene (Reaction 3). The organic peroxide may be detected by measuring the reduction current of the ferricinium ion.

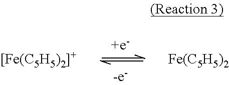

Ferrocene generated by the reduction of the ferricinium ion at the working electrode (detection electrode 10) reenters the enzyme body 3, and can be repetitively used as the mediator 14.

### Example 3

A detector of Example 3 is a detector based on the first embodiment that is capable of detecting formaldehyde, which is a substance that causes sick building syndrome.

The detector 100 of Example 3 includes one type of enzyme body 3 that includes one kind of enzyme 5. In the detector 100 of Example 3, the enzyme 5 is formaldehyde dehydrogenase, and catalyzes an enzyme reaction in which the substrate is formaldehyde, which is also the measurement target substance 6. The detector 100 of Example 3 also uses NAD⁺ as the mediator 14 which functions as another substrate in the enzyme reaction in which formaldehyde is a substrate.

In the detector 100 of Example 3, a solution mixture (χ_{PIL}=AIL/PIL=0.6) of [C₈mIm⁺][TFSA⁻] as AIL and [C₈ImH⁺][TFSA⁻] as PIL is used as the medium 2. [C₈mIm⁺][TFSA⁻] is a hydrophobic ionic liquid, and [C₈ImH⁺][TFSA⁻] is a hydrophilic ionic liquid. [C₈ImH⁺][TFSA⁻] also functions as a cosurfactant.

AOT is added to this solution mixture, and AOT (0.07 M) is dispersed by stirring the solution mixture for 20 hrs. Subsequently, a dilute buffer solution [0.1 M phosphoric acid buffer, pH=7.4] including formaldehyde dehydrogenase as the enzyme 5 is added as an aqueous solution, and the solution mixture is stirred for 1 hr. Therefore, a reversed micelle or microemulsion (W/IL) made of AOT and [C₈ImH⁺][TFSA⁻] including a water pool 4 is prepared in the solution mixture of [C₈mIm⁺][TFSA⁻] and [C₈ImH⁺][TFSA⁻] as the medium 2.

By the abovementioned injection method, the reversed micelle or microemulsion (W/IL) in which formaldehyde dehydrogenase is solubilized in the water pool 4 is formed as the enzyme body 3. The mixture 102 including the enzyme body 3 and medium 2 is thus obtained.

In the detector 100 of Example 3, when formaldehyde as the measurement target substance 6 is introduced to the mixture 102, formic acid is generated due to oxidation of formaldehyde while NAD⁺ as the mediator 14 is reduced into NADH by the enzyme reaction catalyzed by formaldehyde dehydrogenase, which is the enzyme 5 of the enzyme body 3 (Reaction 4).

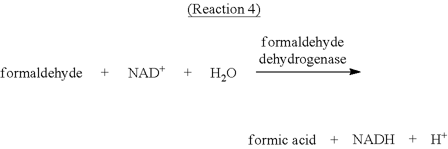

In the detector 100 of Example 3, NADH may be detected using working electrodes (detection electrode 10 and comparison electrode 11) made of, e.g., graphene oxide, by applying, to the working electrodes, a constant potential within a range that is higher than the oxidation potential of NADH, and performing the S1 or S2 measurement mode. At the detection electrode 10, NADH is oxidized into NAD⁺ (Reaction 5). Formaldehyde may be detected by thus measuring the oxidation current of NADH.

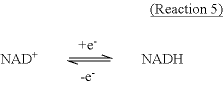

The working electrode is not limited to graphene oxide. For example, an electrode made of a hybrid material including graphene oxide and platinum nanoparticles may be used as the working electrode.

When performing the S1 measurement mode, a carbon electrode made of carbon ink and a platinum pseudo reference electrode may be respectively used as the counter electrode and reference electrode.

Details of the S1 and S2 measurement modes are the same as those described above.

When the concentration of formaldehyde introduced to the mixture 102 of the detector 100 of Example 3 increases, the oxidation current of NADH also increases. A calibration curve indicating the relationship between the concentration and oxidation current of NADH may be prepared in advance, and this calibration curve may be stored as a database in a data processor of the measuring unit 9. By using the calibration curve, quantitative measurement of formaldehyde may be performed based on the detected oxidation current value of NADH.

Furthermore, NAD⁺ generated by the oxidation of NADH at the working electrode (detection electrode 10) reenters the enzyme body 3, and can be repetitively used as the mediator 14 of the enzyme reaction of the enzyme 5.

### Example 4

A detector of Example 4 is a detector based on the first embodiment that is capable of detecting alcohol (ethanol).

The detector 100 of Example 4 has the same arrangement as that of the detector 100 of Example 3, except that alcohol dehydrogenase (ADH) is the enzyme 5.

In the detector 100 of Example 4, when ethanol as the measurement target substance 6 is introduced to the mixture 102, acetaldehyde is generated due to oxidation of ethanol while NAD⁺ as the mediator 14 is reduced into NADH by an enzyme reaction catalyzed by alcohol dehydrogenase, which is the enzyme 5 of the enzyme body 3 (Reaction 6).

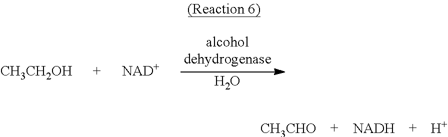

In the detector 100 of Example 4, in a similar manner as in Example 3, ethanol may be detected using working electrodes (detection electrode 10 and comparison electrode 11), by applying a constant potential to the working electrodes, and measuring the oxidation current of NADH by performing the S1 or S2 measurement mode.

As in Example 3, NAD⁺ can be repetitively used as the mediator 14 in the detector 100 of Example 4, as well.

### Example 5

A detector of Example 5 is a detector based on the first embodiment that is capable of detecting glucose.

The detector 100 of Example 5 has the same arrangement as that of the detector 100 of Example 3, except that glucose oxidase (GOD) is the enzyme 5, and ferricyanide (Fe(CN)₆) is the mediator 14.

In the detector 100 of Example 5, when glucose as the measurement target substance 6 is introduced to the mixture 102, gluconolactone is generated due to oxidation of glucose while [Fe(CN)₆]³⁻ as the mediator 14 is reduced into [Fe(CN)₆]⁴⁻ by an enzyme reaction catalyzed by GOD, which is the enzyme 5 of the enzyme body 3 (Reaction 7).

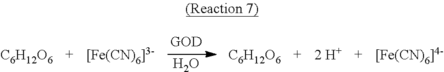

In the detector 100 of Example 5, [Fe(CN)₆]⁴⁻ may be detected by the S1 or S2 measurement mode using working electrodes (detection electrode 10 and comparison electrode 11) made of, e.g., platinum, and applying a constant potential to the working electrodes. At the detection electrode 10, [Fe(CN)₆]⁴⁻ is oxidized into [Fe(CN)₆]³⁻ (Reaction 8). Glucose may be detected by thus measuring the oxidation current of [Fe(CN)₆]⁴⁻. When performing the S1 measurement mode using the platinum working electrode (detection electrode 10), a platinum electrode may be used as the counter electrode, and a platinum pseudo reference electrode may be used as the reference electrode.

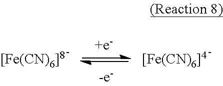

[Fe(CN)₆]³⁻ generated by the oxidation of [Fe(CN)₆]⁴⁻ at the working electrode (detection electrode 10) reenters the enzyme body 3, and can be repetitively used as the mediator 14.

As a modification of Example 5, the detector 100 from which ferricyanide as the mediator 14 is omitted will be explained below.

When the mixture 102 does not include [Fe(CN)₆]³⁻, dissolved oxygen existing in the nonaqueous solvent may be used as the mediator 14. Also, oxygen as the mediator 14 may be replenished from the atmosphere by breathing.

In this modification of Example 5, gluconolactone is generated by oxidation of glucose while oxygen as the mediator 14 is reduced into hydrogen peroxide by the enzyme reaction catalyzed by GOD, which is the enzyme 5 (Reaction 9).

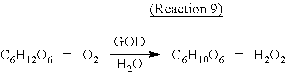

In the detector 100 of the modification of Example 5, hydrogen peroxide generated by the enzyme reaction may be detected by the S1 or S2 measurement mode using working electrodes (detection electrode 10 and comparison electrode 11) made of, e.g., platinum. When a constant potential (640 mV) is applied to the detection electrode 10 as an anode, hydrogen peroxide is oxidized at the detection electrode 10, and oxygen and hydrogen ions are generated (Reaction 10). The oxygen and hydrogen ions are reduced at, e.g., silver electrode as a cathode (counter electrode), and water is generated (Reaction 11). Glucose may be detected by thus directly detecting hydrogen peroxide generated by the enzyme reaction using the detection electrode 10.

H₂O₂→2H⁺+O₂+2e⁻ (Reaction 10)

2H⁺+½O₂+2e⁻→H₂O (Reaction 11)

As described above, hydrogen peroxide generated by the enzyme reaction generates water by the whole of reactions occurring on the surface of the detection electrode 10 as an anode and the surface of the counter electrode (Reaction 12).

H₂O₂→H₂O+½O₂ (Reaction 12)

This water generated as described above reenters the enzyme body 3, thereby replenishing water to the water pool 4. In addition, since the enzyme body 3 is a reversed micelle or microemulsion (W/IL), the amount of water replenished to the water pool 4 is automatically controlled. That is, when the water amount in the water pool 4 reaches the limiting amount of solubilized water of the reversed micelle or microemulsion, extra water generated by the oxidation-reduction reaction is automatically discharged outside from the mixture 102.

Also, as in the modification of Example 5, even when using another kind of enzyme 5 which catalyzes an enzyme reaction that generates hydrogen peroxide, a measurement target substance may be detected by detecting hydrogen peroxide. Examples of enzyme reactions that generate hydrogen peroxide include a cholesterol oxidation reaction catalyzed by cholesterol oxidase, a uric acid oxidation reaction catalyzed by uricase, and a lactic acid oxidation reaction catalyzed by lactate oxidase.

In the detector 100 using such enzyme reactions, water is generated by the oxidation-reduction reaction of the generated hydrogen peroxide at the electrode, and thus water can be replenished to the water pool 4 of the enzyme body 3.

### Example 6

A detector of Example 6 is a detector based on the first embodiment that is capable of detecting glucose.

The detector 100 of Example 6 includes one type of enzyme body 3 that includes one kind of enzyme 5. In the detector 100 of Example 6, the enzyme 5 is glucose oxidase (GOD), and catalyzes an enzyme reaction in which a substrate is glucose, which is the measurement target substance 6. Also, the detector 100 of Example 6 uses a ferricinium ion [Fe(C₅H₅)₂]⁺ as the mediator 14, which is another substrate in the enzyme reaction in which glucose is a substrate.

The enzyme body 3 of Example 6 is manufactured by mixing GOD and a powder of polyvinylalcohol (PVA) until the mixture becomes uniform, and adding a dilute phosphoric acid-citric acid buffer (pH=5), thereby immobilizing GOD by encapsulating it with PVA.

The enzyme bodies 3 thus manufactured are dispersed in a nonaqueous solvent triethylsulfonium bis(trifluoromethylsulfonyl)imide as the medium 2, thereby obtaining the mixture 102 of the medium 2 and enzyme bodies 3. The detector 100 of Example 6 is manufactured using the mixture 102 obtained as described above.

In the detector 100 of Example 6, when glucose as the measurement target substance 6 is introduced to the mixture 102, gluconolactone is generated due to oxidation of glucose while ferricinium ion as the mediator 14 is reduced into ferrocene Fe(C₅H₅)₂ by the enzyme reaction catalyzed by GOD, which is the enzyme 5 of the enzyme body 3 (Reaction 13).

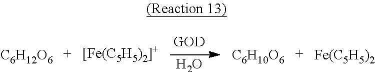

In the detector 100 of Example 6, ferrocene may be detected by the S1 or S2 measurement mode using working electrodes (detection electrode 10 and comparison electrode 11) made of, e.g., platinum, and applying a constant potential (350 mV vs. Pt) to the working electrodes. At the detection electrode 10, ferrocene is oxidized into a ferricinium ion (Reaction 3). Glucose may be detected by thus measuring the oxidation current of ferrocene. When performing the S1 measurement mode by using the platinum working electrode (detection electrode 10), a platinum electrode may be used as a counter electrode, and a platinum pseudo reference electrode may be used as a reference electrode.

The ferricinium ion generated by the oxidation of ferrocene at the working electrode (detection electrode 10) reenters the enzyme body 3, and can be repetitively used as the mediator 14 of the enzyme reaction of the enzyme 5.

### Example 7

A detector of Example 7 is a detector based on the first embodiment that is capable of detecting glucose.

The detector 100 of Example 7 has the same arrangement as that of the detector 100 of Example 6, except that p-benzoquinone is the mediator 14, and the enzyme body 3 is manufactured as follows.

The enzyme body 3 of Example 7 is manufactured by performing modification (inclusive immobilization) of glucose oxidase to a molecular hydrogel as follows.

First, a suspension is prepared by mixing Fmoc-L-lysine (36 mg), Fmoc-L-phenylalanine (38 mg), and sodium carbonate (20 g) (mixing ratio of about 1:1:1.9), then adding 0.9 mL of a phosphoric acid buffer (PBS) (pH=7.4) (104 mg/mL) to the mixture, and stirring the mixture. Then, the suspension is heated to 60° C. while stirring. Since the suspension gels and becomes a transparent molecular hydrogel at 60° C., heating is continued until the suspension becomes completely transparent, thereby forming a molecular hydrogel.

Subsequently, the molecular hydrogel is cooled to 35° C. to 40° C., and glucose oxidase is added to the cooled molecular hydrogel. After stirring, the mixture is cooled to room temperature. The enzyme body 3 of Example 7 is obtained by thus immobilizing glucose oxidase by including it in the molecular hydrogel.

The enzyme bodies 3 obtained as described are dispersed in a nonaqueous solvent triethylsulfonium bis(trifluoromethylsulfonyl)imide as the medium 2, thereby manufacturing the mixture 102 of the medium 2 and enzyme bodies 3.

In the detector 100 of Example 7, when glucose as the measurement target substance 6 is introduced to the mixture 102, gluconolactone is generated due to oxidation of glucose while p-benzoquinone as the mediator 14 is reduced into hydroquinone by an enzyme reaction catalyzed by glucose oxidase, which is the enzyme 5 of the enzyme body 3 (Reaction 14).

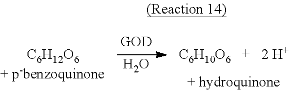

In the detector 100 of Example 7, in a similar manner as in Example 6, hydroquinone may be detected by the S1 or S2 measurement mode using working electrodes (detection electrode 10 and comparison electrode 11) made of, e.g., platinum, and applying, to the working electrodes, a constant potential within a range that is higher than the oxidation potential of hydroquinone. At the detection electrode 10, hydroquinone is oxidized into p-benzoquinone (Reaction 15). Glucose may be detected by thus measuring the oxidation current of hydroquinone. When performing the S1 measurement mode by using the platinum working electrode (detection electrode 10), a platinum electrode may be used as a counter electrode, and a platinum pseudo reference electrode may be used as a reference electrode.

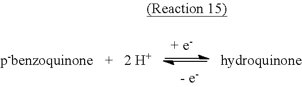

P-benzoquinone generated by the oxidation of hydroquinone at the working electrode (detection electrode 10) reenters the enzyme body 3, and can be repetitively used as the mediator 14.

### Example 8

A detector of Example 8 is a detector based on the first embodiment that is capable of detecting glucose.

The detector of Example 8 includes one type of enzyme body 3 that includes two kinds of enzymes 5 (first and second enzymes). Also, the enzyme body 3 of the detector of Example 8 uses two kinds of mediators (first and second mediators).

In the detector 100 of Example 8, the first enzyme is glucose oxidase (GOD), and catalyzes an enzyme reaction (first enzyme reaction) in which the substrate is glucose, which is the measurement target substance 6.

Oxygen is used as the first mediator. This oxygen as the first mediator is dissolved oxygen existing in a nonaqueous solvent, and can be replenished from the atmosphere by breathing. Gluconolactone (C₆H₁₀O₆) is generated due to oxidation of glucose while oxygen as the first mediator is reduced into hydrogen peroxide by the first enzyme reaction (Reaction 9).

Hydrogen peroxide generated by the first enzyme reaction functions as a substrate of an enzyme reaction (second enzyme reaction) catalyzed by HRP as the second enzyme. In addition, hydroquinone participates as the second mediator in the second enzyme reaction. Hydrogen peroxide is reduced into water, while hydroquinone as the second mediator is oxidized into p-benzoquinone by the second enzyme reaction (Reaction 16).

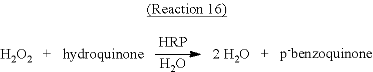

As the medium 2 of Example 8, a solution mixture (χ_{PIL}=AIL/PIL=0.7) of [C₈mIm⁺][TFSA⁻] as AIL and [C₈ImH⁺][TFSA⁻] as PIL is used. [C₈mIm⁺][TFSA⁻] is a hydrophobic ionic liquid, and [C₈ImH⁺][TFSA⁻] is a hydrophilic ionic liquid. [C₈ImH⁺][TFSA⁻] also functions as a cosurfactant.

AOT is added to this solution mixture, and AOT (0.07 M) is dispersed by stirring the solution mixture for 20 hrs. Subsequently, a dilute buffer solution [0.02 M phosphate/borate/acetate, pH=7.0] including GOD as the first enzyme and HRP as the second enzyme is added as an aqueous solvent, and the solution mixture is stirred for 1 hr. Accordingly, a reversed micelle or microemulsion (W/IL) made of AOT and [C₈ImH⁺][TFSA⁻] including a water pool 4 is prepared in the solution mixture of [C₈mIm⁺][TFSA⁻] and [C₈ImH⁺][TFSA⁻] as the medium 2.

GOD (the first enzyme) and HRP (the second enzyme) as enzymes 5 are solubilized in the water pool 4 of the reversed micelle or microemulsion (W/IL) thus obtained.

As described above, when glucose as the measurement target substance 6 is introduced to the mixture 102 in the detector of Example 8, oxygen as the first mediator is reduced into hydrogen peroxide by the first enzyme reaction catalyzed by GOD, which is the first enzyme of the enzyme body 3 (Reaction 9). Hydrogen peroxide is reduced into water while hydroquinone as the second mediator is oxidized into p-benzoquinone by the second enzyme reaction catalyzed by HRP, which is the second enzyme (Reaction 16). If the water amount reaches the limiting amount of solubilized water of the water pool 4, extra water is discharged from the water pool 4 of the reversed micelle.

On the other hand, p-benzoquinone may be detected by the S1 or S2 measurement mode using working electrodes (detection electrode 10 and comparison electrode 11) made of, e.g., platinum, in the same manner as in Example 7. When performing the S1 measurement mode by using the platinum working electrode (detection electrode 10), a platinum electrode may be used as a counter electrode, and a platinum pseudo reference electrode may be used as a reference electrode.

As in Example 7, hydroquinone can be repetitively used as the mediator 14 in the detector 100 of Example 8, as well.

### Example 9

The detector 100 of Example 9 has the same arrangement as that of the detector 100 of Example 8, except that ferrocene Fe(C₅H₅)₂ is used as the second mediator.

In the detector 100 of Example 9, when glucose as the measurement target substance 6 is introduced to the mixture 102, as a result, hydrogen peroxide is reduced into water while ferrocene as the second mediator is oxidized into ferricinium ion [Fe(C₅H₅)₂]⁺ by the second enzyme reaction (Reaction 17).

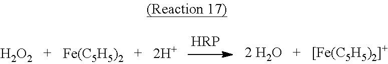

In the detector 100 of Example 9, glucose may be quantitatively measured in a manner similar as in Example 2, by performing the S1 or S2 measurement mode by measuring the reduction current of the ferricinium ion using working electrodes (detection electrode 10 and comparison electrode 11).

### Example 10

A detector of Example 10 is a detector based on the first embodiment that is capable of detecting cholesterol ester and cholesterol.

The detector 100 of Example 10 includes one type of enzyme body 3 that includes three kinds of enzymes 5 (first, second, and third enzymes). In addition, the enzyme body 3 of the detector of Example 10 uses two kinds of mediators (first and second mediators).

In the detector 100 of Example 10, the first enzyme is cholesterol esterase (ChEt), and catalyzes an enzyme reaction (first enzyme reaction) in which the substrate is cholesterol ester, which is the measurement target substance 6. The first enzyme reaction is hydrolysis and requires water. The first enzyme reaction hydrolyzes cholesterol ester, and generates cholesterol and fatty acid (Reaction 18).

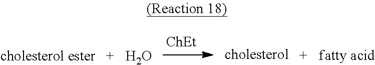

This cholesterol generated by the first enzyme reaction functions as a substrate of a second enzyme reaction catalyzed by cholesterol oxidase (ChOx) as the second enzyme. The second enzyme reaction generates cholestenone by oxidizing cholesterol, and generates hydrogen peroxide by reducing oxygen as the first mediator (Reaction 19). As in Example 8, this oxygen as the first mediator is dissolved oxygen existing in a nonaqueous solvent, and can be replenished from the atmosphere by breathing.

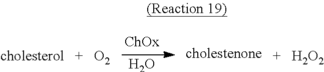

Hydrogen peroxide generated by the second enzyme reaction is reduced into water by a third enzyme reaction catalyzed by HRP as the third enzyme. At the same time, hydroquinone as the second mediator is oxidized into p-benzoquinone (Reaction 16).

In the detector 100 of Example 10, may be detected in a manner similar as in Example 8, by measuring the reduction current of p-benzoquinone.

In addition, since cholesterol is the substrate of the second enzyme reaction in the detector 100 of Example 10, cholesterol itself may be detected as the measurement target substance 6. It is also possible to measure the total amount of cholesterol ester and cholesterol.

In Example 10, the mixture 102 is a gelled mixture 102 manufactured by the following method, unlike in Example 8.

First, the enzyme body 3 is obtained by manufacturing a reversed micelle or microemulsion in which cholesterol esterase (ChEt) as the first enzyme, cholesterol oxidase (ChOx) as the second enzyme, and HRP as the third enzyme are solubilized, by a method similar to that of Example 8.

Then, an ionic liquid solution mixture used in the formation of the enzyme bodies 3 is set at a temperature of 40° C. to 50° C. in a state in which the enzyme bodies 3 are dispersed, and an appropriate amount of a gelatin powder is added to the solution mixture. After that, the solution mixture is vigorously stirred for about 30 min. Subsequently, the solution mixture is cooled to 30° C. while stirring, and kept stirring until the solution becomes very thick and uniform. The obtained suspension is left to stand at room temperature until the solution becomes a transparent gel.

In the abovementioned treatment process, gelatin enters the water pool 4 of the enzyme body 3 (the reversed micelle or microemulsion), and gels there. Furthermore, since gelatin having gelled in the water pool 4 forms an intermolecular network, the whole mixture 102 including the enzyme bodies 3 gels. In addition, since the suspension is left to stand at room temperature, refolding of proteins (gelatin, glucose oxidase, and HRP) that had been thermally denatured by heating may be performed.

When measuring the total amount of cholesterol ester and cholesterol by performing the S2 measurement mode, an ionic liquid gel (ionogel) in which cholesterol esterase (ChEt), cholesterol oxidase (ChOx), and HRP, which are respectively the first, second, and third enzymes, are not solubilized is used as the medium 2 that is disposed in contact with the comparison electrode 11. This ionic liquid gel is manufactured using a solution mixture of [C₈mIm⁺][TFSA⁻] as AIL and [C₈ImH⁺][TFSA⁻] as PIL, AOT as an anionic surfactant, a buffer solution [0.1 M phosphoric acid buffer, pH=7.4], and gelatin, in a manner similar to the ionic liquid gel in contact with the detection electrode 10.

### Example 11

A detector of Example 11 is a detector based on the first embodiment that is capable of detecting cholesterol ester and cholesterol.

The detector 100 of Example 11 includes two types of enzyme bodies 3 (first and second enzyme bodies), and each type of enzyme body includes one of different kinds of enzymes 5 (first and second enzymes). In addition, the first enzyme body of the detector of Example 11 uses a mediator 14 (a first mediator) which functions as a substrate of an enzyme reaction catalyzed by the first enzyme included therein. The second enzyme body uses a mediator 14 (a second mediator) which functions as a substrate of an enzyme reaction catalyzed by the second enzyme included therein. The first and second mediators are different kinds of mediators as described later.

As described below, the arrangement of the detector 100 of Example 11 is practically the same as that of the detector 100 of Example 8, except that the reaction fields of the first and second enzyme reactions are divided into the first and second enzyme bodies.

In the detector 100 of Example 11, the first enzyme is glucose oxidase (GOD), and catalyzes an enzyme reaction (the first enzyme reaction) in which the substrate is glucose, which is the measurement target substance 6, as in Example 8.

Also, oxygen is used as the first mediator as in Example 8.

In the detector 100 of Example 11, the second enzyme is HRP as in Example 8. Therefore, the second enzyme reaction in the detector 100 of Example 11 is the same as the second enzyme reaction of Example 8.

The medium 2 of Example 11 is prepared by the same method as in Example 8, except that the ratio of AIL to PIL is adjusted such that χ_{PIL}=AIL/PIL=0.6 in a solution mixture of AIL and PIL as the medium 2.

Except that this medium 2 and as an aqueous solvent a dilute buffer solution [0.1 M phosphoric acid buffer, pH=7.4] including glucose oxidase (GOD) as the first enzyme is used, in a manner similar as in Example 8, a reversed micelle or microemulsion (W/IL) made of AOT and [C₈ImH⁺][TFSA⁻], in which GOD as the first enzyme is solubilized in the water pool 4, i.e., the first enzyme body dispersed in the medium 2 of Example 11, is formed.

Separately, except that a dilute buffer solution [0.1 M phosphoric acid buffer, pH=7.4] including HRP as the second enzyme is used as an aqueous solvent, by a method similar to the formation of the first enzyme body, a reversed micelle or microemulsion (W/IL) made of AOT and [C₈ImH⁺][TFSA⁻], in which HRP as the second enzyme is solubilized in the water pool 4, i.e., namely, the second enzyme body dispersed in the medium 2 of Example 11 is formed.

The mixture 102 of Example 11 is manufactured by mixing the medium 2 in which the first enzyme bodies are dispersed and the medium 2 in which the second enzyme bodies are dispersed.

In the detector 100 of Example 11, when glucose as the measurement target substance 6 is introduced to the mixture 102, enzyme reactions (the first and second enzyme reactions) similar to Example 8 proceed and generate p-benzoquinone. Unlike in Example 8, however, the first and second enzyme reactions respectively proceed in the first and second enzyme bodies in Example 11. That is, hydrogen peroxide generated by the first enzyme reaction leaves the first enzyme body, enters the second enzyme body, and there becomes reduced by the second enzyme reaction.

Except the foregoing, the detector 100 of Example 11 has the same arrangement as that of the detector 100 of Example 8, and may detect glucose in a manner similar as in the detector 100 of Example 8.

### Example 12

The detector 100 of Example 12 has the same arrangement as that of the detector 100 of Example 11, except that ferrocene Fe(C₅H₅)₂ is used as the second mediator.

In the detector 100 of Example 12, glucose may be quantitatively measured by performing the S1 or S2 measurement mode in a manner similar as in Example 2 by measuring the reduction current of a ferricinium ion [Fe(C₅H₅)₂]⁺ using working electrodes (detection electrode 10 and comparison electrode 11).

### Example 13

A detector of Example 13 is a detector based on the first embodiment that is capable of detecting acetone.

The detector 100 of Example 13 includes one type of enzyme body 3 that includes one kind of enzyme 5. In the detector 100 of Example 13, the enzyme 5 is secondary alcohol dehydrogenase (S-ADH), and catalyzes an enzyme reaction in which the substrate is acetone, which is the measurement target substance 6. Also, the detector 100 of Example 13 uses NADH as the mediator 14 in the enzyme reaction in which acetone is a substrate.

In Example 13, 1-butyl-3-methylimidazolium hexafluorophosphate ([bmim][PF₆]), which is an ionic liquid, is used as the medium 2.

Brij-35 as a surfactant is added to [bmim][PF₆], and Brij-35 is dispersed in [bmim][PF⁶] by stirring, thereby forming a reversed micelle. Then, an appropriate amount of 100 mM phosphoric acid buffer (100 mM PBS, pH=7.8) as buffer solution is added as an aqueous solution, and the solution mixture is stirred. Consequently, a reversed micelle or microemulsion (water/brij-35 (0.5 M)/[bmim][PF₆]) made of brij-35 and [bmim][PF₆] including a water pool 4 is prepared.

The enzyme body 3 is manufactured by solubilizing S-ADH as the enzyme 5 into the water pool 4 of water/brij-35 (0.5 M)/[bmim][PF₆] thus obtained. In addition, in Example 13, the amount of water is so adjusted that the water content in the water pool 4 is, e.g., ω₀=17.

Alternatively, reversed micelle (water/brij-35 (0.5 M)/[bmim][PF₆]) in which S-ADH is solubilized may be manufactured when adding brij-35 as a surfactant to [bmim][PF₆] as the medium 2 and stirring the mixture, by adding a 100 mM phosphoric acid buffer (100 mM PBS, pH=7.8) including an appropriate amount of S-ADH while stirring, and sufficiently stirring the mixture.

In the detector 100 of Example 13, when acetone as the measurement target substance 6 is introduced to the mixture 102, isopropanol is generated due to reduction of acetone while NADH as the mediator 14 is oxidized into NAD⁺ by the enzyme reaction catalyzed by S-ADH, which is the enzyme 5 of the enzyme body 3 (Reaction 20).

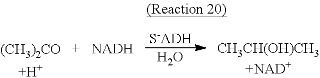

In the detector 100 of Example 13, NAD⁺ may be detected by the S1 or S2 measurement mode using working electrodes (detection electrode 10 and comparison electrode 11) made of, e.g., graphene oxide, and applying a constant potential to the working electrodes. At the detection electrode 10, NAD⁺ is reduced into NADH (Reaction 5). Acetone may be detected by thus measuring the reduction current of NAD⁺.

Details of the S1 and S2 measurement modes in Example 13 are the same as those of Example 2, except that the potential applied to the working electrodes is different, and that the reduction current of NAD⁺ is measured.

NADH generated by the reduction of NAD⁺ at the working electrode (detection electrode 10) reenters the enzyme body 3, and can be repetitively used as the mediator 14 of the enzyme reaction of the enzyme 5.

It is also possible to perform chronoamperometry (CA) measurement of NADH by using a microelectrode. This CA measurement performed on NADH using the microelectrode is a method of measuring a steady-state current generated by the oxidation of NADH. The diameter of the microelectrode is, e.g., 50 μm, and a carbon printed electrode coated with graphene oxide similar to that of Example 3 may be used as a graphene oxide microelectrode. In this case, a silver electrode may be used as a reference electrode as in Example 2. Also, a carbon electrode may be used as a counter electrode.

Furthermore, cyclic voltammetry (CV) measurement of NADH may be performed by using the microelectrode.

In the detector 100 of Example 13, Acetone may be detected also by measuring NADH using an optical measurement method.

The concentration of NADH in the mixture 102 held in the measuring cell 101 of Example 13 may be measured based on the Lambert-Beer law by measuring the absorbance of the mixture 102 at a wavelength of, e.g., 340 nm.

As described above, when acetone is introduced to the mixture 102, the enzyme reaction oxidizes NADH into NAD⁺. A decrease in concentration of NADH caused by the enzyme reaction may be detected by measuring the absorbance of the mixture 102 at a wavelength of 340 nm. Acetone may be detected and measured based on this decrease in NADH concentration in the mixture 102.

### Example 14

The detector 100 of Example 14 is a detector based on the first embodiment that is capable of detecting alcohol (ethanol) by an optical measurement method.

The measuring cell 101 of Example 14 holds the mixture 102 including the enzyme body 3 including alcohol oxidase and peroxidase (HRP) as enzymes 5, a nonaqueous solvent 1-butyl-3-methylimidazolium chloride (bmimCl) as the medium 2, and 2,6-dichloroindophenol sodium salt hydrate (DCIP) as a dye.

This measuring cell of Example 14 is manufactured as follows.

A solution mixture is obtained by adding 1 g of Avicel® (a cellulose powder manufactured by FMC) to a 0.01 M phosphoric acid buffer solution (1 mL) including 3 mg/mL of alcohol oxidase, 0.02 mg/mL of HRP, and 7 mM of DCIP. Then, the solution mixture is subjected to an air flow at room temperature until the water content becomes 36%, thereby forming enzyme bodies 3. A mixture 102 is obtained by mixing the enzyme bodies 3 thus obtained and bmimCl at a predetermined mixing ratio. The measuring cell 101 is formed by putting the mixture 102 into a main cell member 1.

The concentration of DCIP in the mixture 102 held in the measuring cell 101 of Example 14 may be measured based on the Lambert-Beer law by measuring the absorbance of the mixture 102 at a wavelength of, e.g., 605 nm.

When alcohol (ethanol) as the measurement target substance 6 is introduced to the measuring cell 101 of Example 14, enzyme reactions catalyzed by alcohol oxidase and HRP as the enzymes 5 decompose DCIPₒₓ in the oxidized form into a decomposition product (DCIP_{decomp}). More specifically, ethanol is oxidized into acetaldehyde while hydrogen peroxide is generated by the enzyme reaction catalyzed by alcohol oxidase (Reaction 21). This hydrogen peroxide decomposes DCIPₒₓ by an enzyme reaction catalyzed by HRP (Reaction 22).

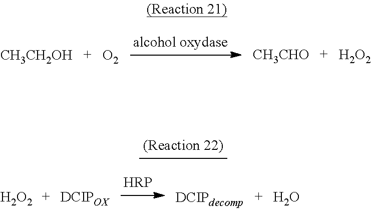

A decrease in concentration of DCIPₒₓ caused by the enzyme reaction may be detected by measuring the absorbance of the mixture 102 at a wavelength of 605 nm. Alcohol (ethanol) may be detected by thus measuring the change in absorbance of DCIPₒₓ.

A nonaqueous solvent 1-butyl-3-methylimidazolium hexafluorophosphate [bmim][PF₆]) may also be used as the medium 2 of Example 14.

Practical examples according to the second embodiment will be explained below.

### Example 15

The detector 200 of Example 15 is a detector based on the second embodiment that is capable of detecting a nerve gas (sarin or VX).

The detector 200 of Example 15 includes one type of enzyme body 3 that includes one kind of enzyme 5, and further includes a substrate 15. In the detector 200 of Example 15, the enzyme 5 is acetylcholinesterase (AChE), and the substrate 15 is acetylthiocholine chloride (ATChCl). Also, the measurement target substance 6 to be detected by the detector 200 of Example 15 may be a nerve gas (sarin or VX), and the gas is an inhibitor of an enzyme reaction catalyzed by AChE, in which ATChCL is the substrate.

Triethylsulfonium bis(trifluoromethylsulfonyl)imide as a nonaqueous solvent may be used as the medium 2.

The enzyme body 3 may be manufactured as follows.

AChE as the enzyme 5 and 5% albumin (bovine serum albumin; BSA) are dispersed in an aqueous sol of porous spherical silica particles having mesopores (average particle size: 0.3 μm, pore size: 16 nm) (phosphate buffered saline; PBS, pH=7.4). Accordingly, the enzyme bodies 3 by immobilizing AChE to the porous spherical silica particles having mesopores are formed. A sol of the enzyme bodies 3 thus obtained is dispersed in the abovementioned medium 2, thereby obtaining the mixture 202 including the enzyme bodies 3 and medium 2. The porous spherical silica particles having hydrophilic mesopores are hygroscopic and hence can further absorb water from the atmosphere, therefore water can automatically be replenished to the enzyme body 3.

In the detector 200 of Example 15, a powder of ATChCl as the substrate of the enzyme reaction catalyzed by the enzyme 5 (AChE) is also dispersed in the medium 2.

ATChCl as the substrate 15 is hydrolyzed by the enzyme reaction catalyzed by AChE, which is the enzyme 5 included in the enzyme body 3, thereby generating, e.g., thiocholine (TCh) (Reaction 23).

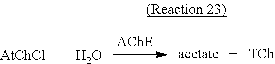

In the detector 200 of Example 15, TCh may be measured by the S1 or S2 measurement mode using a detection electrode 10 made of, e.g., platinum. This measurement of TCh by the S1 or S2 measurement mode may be performed in the same manner as in the measurement of PNP as a product of the enzyme reaction in Example 1.

In the detector 200 of Example 15, when a nerve gas as the measurement target substance 6 is introduced to the mixture 202, the enzyme reaction catalyzed by AChE as the enzyme 5 included in the enzyme body 3, i.e., the hydrolysis of ATChCl is inhibited. As a consequence, the generation amount of TCh decreases.

This decrease in TCh may be detected by the above-described TCh measurement. The nerve gas is detected based on the decrease of TCh thus detected. Quantitative measurement of nerve gas may be performed by using a database constructed by, e.g. forming a calibration curve beforehand.

As another nerve gas detecting method, it is also possible to detect a nerve gas by using a detector including, e.g., an ISFET as the detector 200 of Example 15, and measuring a change in pH of the medium 2 due to a hydrolysate (e.g., acetate) of ATChCl. Alternatively, a nerve gas may be detected by measuring a change in pH of the medium 2 by using potentiometry. When measuring a change in pH of the medium 2 as described above, a sol including no phosphoric acid buffer is used as the water solvent based sol of the porous spherical silica particles used in the formation of the enzyme body 3.

### Example 16

The detector 200 of Example 16 is a detector based on the second embodiment capable of detecting a nerve gas (sarin or VX).

The detector 200 of Example 16 includes one type of enzyme body 3 that includes two kinds of enzymes 5 (first and second enzymes), and further includes the substrate 15. In the detector 200 of Example 16, the first enzyme is cholinesterase (ChE), and catalyzes an enzyme reaction (first enzyme reaction) in which acetylcholine chloride (ACh) is the substrate 15.

ACh as the substrate 15 generates choline (Ch) and an organic acid (RCOOH) by the first enzyme reaction catalyzed by ChE as the first enzyme (Reaction 24). The first enzyme reaction is hydrolysis and hence requires water.

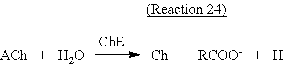

Ch generated by the first enzyme reaction functions as a substrate of an enzyme reaction (second enzyme reaction) catalyzed by choline oxidase (ChO) as the second enzyme. The second enzyme reaction is hydrolysis and hence requires water. Also, oxygen participates as the mediator 14 in the second enzyme reaction. This oxygen as the mediator 14 is dissolved oxygen existing in a nonaqueous solvent, and may be replenished from the atmosphere by breathing.

ACh generates Ch by the enzyme reaction of the first enzyme 5 (ChE). Generated Ch functions as a substrate of ChO as the second enzyme 5. Ch generated by the first oxidation reaction is hydrolyzed by the second enzyme reaction, and oxygen as the mediator 14 is reduced to generate hydrogen peroxide (Reaction 25).

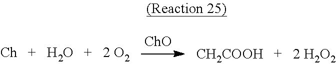

The mixture 202 including the medium 2 that includes a nonaqueous solvent and the enzyme body 3 is formed as follows.

As the medium 2 of Example 16, a solution mixture of AIL and PIL similar to that of the medium 2 of Example 3 is used. A reversed micelle or microemulsion (W/IL) dispersed in the medium 2 is manufactured in the same manner as in Example 3 except that 5% BSA is used as an aqueous solution. The enzyme body 3 in which ChE as the first enzyme and ChO as the second enzyme are solubilized in the water pool 4 of the reversed micelle or microemulsion (W/IL) is manufactured.

Also, a powder of ACh as the substrate of the first enzyme reaction is dispersed in the mixture 202 including the medium 2 and enzyme body 3 obtained as described above.

Hydrogen peroxide generated by the second enzyme reaction may be detected by the S1 or S2 measurement mode using working electrodes (detection electrode 10 and comparison electrode 11) made of, e.g., platinum. When a constant potential (640 mV) is applied to the detection electrode 10 as an anode, hydrogen peroxide is oxidized at the detection electrode 10, thereby generating oxygen and hydrogen ions. These oxygen and hydrogen ions are reduced at, e.g., a silver electrode as a cathode (counter electrode), thereby generating water. The generated water can reenter the enzyme body 3, and participate in the enzyme reactions (first and second enzyme reactions).

In Example 16 as described above, hydrogen peroxide generated by the enzyme reaction in the enzyme body 3 generates water by further reacting at the electrode, so water can be regenerated in the system of the detector 200. This makes it possible to uninterruptedly supply water necessary for the hydrolysis enzyme reaction.

A nerve gas (sarin or VX) as the measurement target substance 6 is an inhibitor of the first enzyme reaction catalyzed by ChE. In the detector 200 of Example 16, when a nerve gas as the measurement target substance 6 is introduced to the mixture 202, the first enzyme reaction catalyzed by ChE as the first enzyme, i.e., the hydrolysis of ACh is inhibited. Consequently, the generation amount of Ch decreases, and thus decreases the generation amount of hydrogen peroxide as a product of the second enzyme reaction in which Ch is the substrate.

In the detector 200 of Example 16, decrease in hydrogen peroxide may be detected by measuring hydrogen peroxide by the above-described S1 or S2 measurement mode using the detection electrode 10. The nerve gas is detected based on the decrease in hydrogen peroxide thus detected. Quantitative measurement of nerve gas may be performed by using a database constructed by, e.g., forming a calibration curve beforehand.

### Example 17

The detector 200 of Example 17 is a detector based on the second embodiment that is capable of detecting a nerve gas (sarin or VX).

The detector 200 of Example 17 includes two types of enzyme bodies 3 (first and second enzyme bodies), and each type of enzyme body 3 includes one of different kinds of enzymes 5 (first and second enzymes). The detector 200 of Example 17 further includes the substrate 15.

The detector 200 of Example 17 has the same arrangement as that of the detector 200 of Example 16, except that the detector 200 of Example 17 includes the first enzyme body including ChE as the first enzyme, and the second enzyme body including Cho as the second enzyme.

In the detector 200 of Example 17, the first enzyme reaction proceeds in the first enzyme body, and the second enzyme reaction proceeds in the second enzyme body, unlike in Example 16. That is, Ch generated by the first enzyme reaction leaves the first enzyme body, enters the second enzyme body, and there becomes oxidized by the second enzyme reaction.

Except the foregoing, the detector 200 of Example 17 has the same arrangement as that of the detector 200 of Example 16, and may detect a nerve gas in a similar manner as in the detector 200 of Example 16.

### Example 18

The detector 200 of Example 18 is a detector based on the second embodiment that is capable of detecting a nerve gas (sarin or VX).

The detector 200 of Example 18 includes one type of enzyme body 3 that includes three kinds of enzymes 5 (first, second, and third enzymes), and further includes the substrate 15. In addition, two kinds of mediators (first and second mediators) are used in the enzyme body 3 of the detector of Example 18.

In the detector 200 of Example 18, the first and second enzymes are respectively ChE and ChO, as in Example 16. First and second enzyme reactions in Example 18 are also the same as those in Example 16, and the substrate of each enzyme reaction is the same as that in Example 16. Furthermore, oxygen participates as the first mediator in the second enzyme reaction in Example 18, as well.

The enzyme body 3 of Example 18 further includes HRP as the third enzyme. HRP as the third enzyme catalyzes an enzyme reaction (third enzyme reaction) in which the substrate is hydrogen peroxide generated by the second enzyme reaction. In Example 18, hydroquinone as the second mediator also participates in the third enzyme reaction.

The enzyme body 3 of Example 18 is manufactured in the same manner as in Example 10, except that the first enzyme is ChE and the second enzyme is ChO.

In Example 18, water can be generated in the enzyme body 3 by using HRP. The water thus regenerated can be used in the first and second enzyme reactions.

In the detector 200 of Example 18, a decrease in p-benzoquinone may be detected by measuring the reduction current of p-benzoquinone in the same manner as in Example 8, and a nerve gas may be detected based on the decrease.

### Example 19

The detector 200 of Example 19 is a detector based on the second embodiment that is capable of detecting a nerve gas (sarin or VX).

The detector 200 of Example 19 includes three types of enzyme bodies 3 (first, second, and third enzyme bodies), and each type of enzyme body 3 includes one of different kinds of enzymes 5 (first, second, and third enzymes). The detector 200 of Example 19 further includes a substrate 15.

The detector 200 of Example 19 has the same arrangement as that of the detector 200 of Example 18, except that the detector 200 of Example 19 includes the first enzyme body including ChE as the first enzyme, the second enzyme body including ChO as the second enzyme, and the third enzyme body including HRP as the third enzyme.

In the detector 200 of Example 19, first, second, and third enzyme reactions respectively proceed in the first, second, and third enzyme bodies, unlike in Example 18. That is, Ch generated by the first enzyme reaction leaves the first enzyme body, enters the second enzyme body, and there becomes oxidized by the second enzyme reaction. Also, hydrogen peroxide generated by the second enzyme reaction leaves the second enzyme body, enters the third enzyme body, and there becomes reduced by the third enzyme reaction.

Except the foregoing, the detector 200 of Example 19 has the same arrangement as that of the detector 200 of Example 16, and may detect nerve gas in a manner similar to the detector 200 of Example 18.

### Example 20

The detector 200 of Example 20 has the same arrangement as that of the detector 200 of Example 18, except that the mixture 202 including the medium 2 and enzyme body 3 is gelled by the same method as that of Example 10. The detector 200 of Example 20 may detect nerve gas in a manner similar to the detector 200 of Example 18.

### Example 21

The detector 200 of Example 21 is a detector based on the second embodiment that is capable of detecting a heavy metal ion.

The detector 200 of Example 21 has the same arrangement as that of the detector 100 of Example 2, except that the mixture 202 held in the main cell member 1 of the measuring cell 201 includes hydrogen peroxide as the substrate 15.

When no heavy metal ion is introduced, by an enzyme reaction catalyzed by HRP, normally, hydrogen peroxide is reduced into water, while at the same time, ferrocene as a mediator is oxidized into ferricinium ion (Reaction 17).

Heavy metal ions such as lead, cadmium, and mercury ions are inhibitors of the enzyme reaction catalyzed by HRP as the enzyme 5. When a heavy metal ion as the measurement target substance 6 is introduced to the mixture 202 held in the measuring cell 201, the reduction reaction of hydrogen peroxide, which is the enzyme reaction catalyzed by HRP, is inhibited. Consequently, the generation amount of ferricinium ions [Fe(C₅H₅)₂]⁺ generated by the oxidation reaction of ferrocene as the mediator decreases. Accordingly, a heavy metal ion may be detected by detecting this decrease in ferricinium ions [Fe(C₅H₅)₂]⁺.

In the detector of Example 21, decrease in ferricinium ions may be measured in a manner similar as in Example 1 using working electrodes (detection electrode 10 and comparison electrode 11) made of, e.g., platinum, by applying a constant potential to the working electrodes, and detecting ferricinium ions by performing the S1 or S2 measurement mode.

Ferrocene generated by the reduction of ferricinium ions at the working electrode (detection electrode 10) reenters the enzyme body 3, and can be repetitively used as the mediator 14.

A practical example according to the third embodiment will be explained below.

### Example 22

A detector of Example 22 is a detector based on the third embodiment capable of detecting trinitrotoluene (TNT) as an explosive.

The detector of Example 22 includes a sampling unit capable of sublimating TNT by heating a sample to be measured including TNT as the measurement target substance 6 at 60° C. The vapor of TNT obtained by the sampling unit is introduced to the mixture in the measuring cell and measured.

The mixture of Example 22 includes one type of enzyme body 3 that includes one kind of enzyme 5. In Example 22, the enzyme 5 is nitroreductase (NTR), and catalyzes an enzyme reaction in which the substrate is TNT, which is the measurement target substance 6. In addition, as another substrate of the enzyme reaction in which TNT is a substrate, NADH is used as the mediator 14.

In Example 22, an ionic liquid 1-butyl-3-methylimidazolium hexafluorophosphate ([bmim][PF₆]) is used as the medium 2.

Brij-35 as a surfactant is added to [bmim][PF₆] and dispersed in [bmim][PF₆] by stirring, thereby forming a reversed micelle. Then, an appropriate amount of 100 mM phosphoric acid buffer (100 mM PBS, pH=7.0) is added as a buffer solution as an aqueous solution, and the solution mixture is stirred, thereby manufacturing a reversed micelle or microemulsion (water/brij-35 (0.5 M)/[bmim][PF₆]) made of brij-35 and [bmim][PF₆], and including a water pool 4.

The enzyme body 3 is manufactured by solubilizing NTR as the enzyme 5 in the water pool 4 of the water/brij-35 (0.5 M)/[bmim][PF₆] thus obtained. In addition, in Example 22, the amount of water is adjusted such that the water content in the water pool 4 is, e.g., ω₀=17.

In the measuring cell of Example 22, when TNT is introduced from the sampling unit to the mixture in the main cell member, TNT is reduced while NADH as the mediator 14 is oxidized into NAD⁺ by the enzyme reaction catalyzed by NTR, which is the enzyme 5 (Reaction 26).

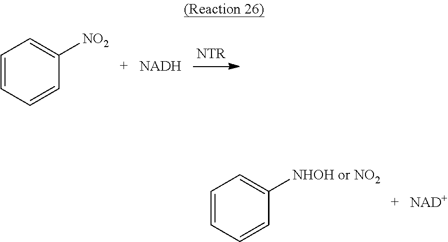

In the detector of Example 22, TNT may be detected by measuring NAD⁺ by an electrochemical or optical measurement method in the same manner as in Example 13.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A measuring cell comprising:
a main cell member; and
a mixture supported by or held in the main cell member, and including a nonaqueous solvent-including medium and one or more enzyme body, the one or more enzyme body being selected from the group consisting of an enzyme, a first composite including an enzyme and a molecular aggregate that includes a dispersant, a microcapsule including an enzyme-including core and a shell covering the core, a cell including an enzyme, a microorganism including an enzyme, and a second composite including an enzyme and a support immobilizing the enzyme.

2. The cell according to claim 1, wherein at least a part of the one or more enzyme body is hygroscopic or includes water in contact with the enzyme, or the enzyme included in at least a part of the one or more enzyme body catalyzes a reaction that generates water, or catalyzes a reaction that generates a compound which is decomposed into water and another compound by a redox reaction.

3. The cell according to claim 1, wherein the nonaqueous solvent includes an ionic liquid.

4. The cell according to claim 1, wherein the mixture further includes a substrate, and the substrate is a reactant of a reaction catalyzed by the enzyme included in at least a part of the one or more enzyme body.

5. The cell according to claim 1, wherein the mixture is a gel.

6. The cell according to claim 1, wherein the one or more enzyme body includes the first composite, and the molecular aggregate includes one or more of a reversed micelle, a reverse wormlike micelle, liposome, vesicle, a microemulsion, a larger emulsion, a bicontinuous microemulsion, a monodispersed single emulsion, a double emulsion, and a multilayered emulsion.

7. The cell according to claim 1, wherein the one or more enzyme body includes the microcapsule, the shell includes a gel or a polymeric material, and the core includes a molecular aggregate that includes a dispersant, a cell, or a microorganism.

8. The cell according to claim 1, wherein the one or more enzyme body includes the microcapsule, and the shell includes a porous hydrophilic membrane.

9. The cell according to claim 1, wherein the one or more enzyme body includes the second composite, and the support includes one or more material selected from the group consisting of a water-absorbing material, a hydrogel, a silica gel, a polymer gel, a molecular gel, a nanofiber gel, an inorganic material having a nanoporous structure, a cyclodextrin/polymer, a nanogel, a nanofiber, a porous spherical silica particle having a mesopore, and a polymeric material.

10. The cell according to claim 1, wherein the enzyme included in at least a part of the one or more enzyme body includes one or more enzyme selected from the group consisting of a hydrolytic enzyme, a redox enzyme, synthase, transferase, an elimination enzyme, a modified enzyme, a protein crosslinking enzyme, a mutation enzyme, an artificial enzyme, a crosslinking enzyme, an antibody enzyme, lyase, ligase, and a crystallized enzyme.

11. A detector comprising:
the measuring cell of claim 1; and
a measuring unit configured to measure an electrical property or an electrochemical property of the mixture
wherein the measuring cell further comprises one or more electrodes disposed in contact with the mixture.

12. An analysis device comprising:
the detector of claim 11; and
a sampling unit including at least one of a vaporizer configured to vaporize a measurement target substance included in a sample to be measured by laser irradiation, UV irradiation, gas spraying, ultrasonic irradiation, heating, and voltage application, and an ionization source configured to ionize the measurement target substance.
